(19) United States
(12) Patent Application Publication
(10) Pub. No.: US 2009/0215998 A1
(43) Pub. Date: Aug. 27, 2009

(54) DEVICES AND METHODS FOR HIGH-PRESSURE REFOLDING OF PROTEINS

(75) Inventors: Jeffrey D. Antman, Louisville, CO (US); Ryan L. Crisman, Golden, CO (US); Lyndal K. Hesterberg, Boulder, CO (US); Robert Nelson, Arvada, CO (US); Theodore W. Randolph, Niwot, CO (US); Matthew B. Seefeldt, Boulder, CO (US)

Correspondence Address: MORRISON & FOERSTER LLP, 755 PAGE MILL RD, PALO ALTO, CA, 94304-1018, US

(73) Assignee: BaroFoid, Inc.

(21) Appl. No.: 12/094,398

(22) PCT Filed: Nov. 21, 2006
(86) PCT No.: PCT/US06/45297

§ 371 Date: Nov. 21, 2008

Related U.S. Application Data
(60) Provisional application No. 60/739,094, filed on Nov. 21, 2005.

(51) Int. Cl. C07K 1/00; B01L 3/00

(52) U.S. Cl. 530/427; 422/102

(57) ABSTRACT

Disclosed are devices for holding samples, particularly liquid samples, during high-pressure treatment. The devices enable a variety of functions, such as high-throughput screening of samples in multi-compartment device embodiments, and adjustment of solution conditions during high-pressure treatment. The devices are designed to maintain integrity during the high-pressure conditions, and are optionally substantially impermeable to oxygen.

### CROSS REFERENCE TO RELATED APPLICATIONS

This application claims priority benefit of U.S. Provisional Patent Application No. 60/739,094, filed Nov. 21, 2005. The entire content of that application is hereby incorporated by reference herein.

### TECHNICAL FIELD

This invention pertains to devices, such as containers, multiwell plates, and systems for pumping fluids to containers and multiwell plates, designed for operation at high hydrostatic pressure. The invention also pertains to methods of using the devices for refolding of proteins under high pressure.

### BACKGROUND

Many proteins are valuable as therapeutic agents. Such proteins include human growth hormone, which is used to treat abnormal height when insufficient growth hormone is produced in the body, and interferon-gamma, which is used to treat neoplastic and viral diseases. Protein pharmaceuticals are often produced using recombinant DNA technology, which can enable production of higher amounts of protein than can be isolated from naturally-occurring sources, and which avoids contamination that often occurs with proteins isolated from naturally-occurring sources.

Proper folding of a protein is essential to the normal functioning of the protein. Improperly folded proteins are believed to contribute to the pathology of several diseases, including Alzheimer's disease, bovine spongiform encephalopathy (BSE, or “mad cow” disease) and human Creutzfeldt-Jakob disease (CJD), and Parkinson's disease; these diseases serve to illustrate the importance of proper protein folding.

Several proteins of therapeutic value in humans, such as recombinant human growth hormone and recombinant human interferon gamma, can be expressed in bacteria, yeast, and other microorganisms. While large amounts of proteins can be produced in such systems, the proteins are often misfolded, and often aggregate together in large clumps called inclusion bodies. The proteins cannot be used in the misfolded, aggregated state. Accordingly, methods of disaggregating and properly refolding such proteins have been the subject of much investigation.

One method of refolding proteins uses high pressure on solutions of proteins in order to disaggregate, unfold, and properly refold proteins. Such methods are described in U.S. Pat. No. 6,489,450, U.S. Patent Application Publication No. 2004/0038333, and International Patent Application WO 02/062827. Those disclosures indicated that certain high-pressure treatments of aggregated proteins or misfolded proteins resulting in recovery of disaggregated protein retaining biological activity (i.e., the protein was properly folded, as is required for biological activity) in good yields. U.S. Pat. No. 6,489,450, U.S. 2004/0038333, and WO 02/062827 are incorporated by reference herein in their entireties.

As indicated in U.S. 2004/0038333, empirical screening procedures are sometimes required to determine the optimal refolding conditions for a protein. Thus there is a need for suitable equipment which can be used in methods to rapidly determine the optimal conditions, such as multiwell plates, disposable single-sample containers, and devices for mixing solutions under high pressure in order to change solution conditions under high pressure.

96-well plates (typically with an 8×12 arrangement of wells) are commonly used in high-throughput screening in biology and biochemistry. However, current commercially available plates are not suitable for high-pressure applications (e.g., 250 bar and higher). The present invention provides such equipment which is suitable for use under high pressure.

Single-sample containers currently used for high-pressure studies also suffer from drawbacks. Containers made from materials such as low-density polyethylene and polypropylene allow significant mass transfer of oxygen under high pressures. For reactions which are sensitive to the redox environment of the solution, such oxygen transfer is highly undesirable. The present invention also provides equipment which reduces or eliminates oxygen mass transfer through the walls of the container when desired.

Yet another drawback of currently used equipment is that solution conditions cannot be adjusted during the high pressure treatment. The present invention provides equipment which allows changes in solution conditions during high pressure treatment, by enabling manipulation of various containers and solutions while the containers and solutions are inside the high pressure apparatus.

### DISCLOSURE OF THE INVENTION

The invention embraces single-sample holding devices, multi-sample holding devices, and solution exchange devices suitable for use at high pressure. In certain embodiments, the devices are fabricated from polymers, which allows relatively low cost fabrication of the devices. This also allows for injection molding of the devices for convenient fabrication. In certain embodiments, the devices can be disposable for ease of use. The solution exchange devices permit changing of solution conditions of the sample while the sample is maintained under high pressure. In optional embodiments, the devices can be made from materials which are substantially oxygen-impermeable.

In one embodiment, the invention embraces a multi-sample holding device comprising at least two compartments for receiving liquid samples, wherein the device maintains the compartments as substantially closed systems when subjected to high pressure.

In another embodiment, the invention embraces a multi-sample holding device comprising: a) a body made from a material that maintains integrity under high pressure; and b) a plurality of sample compartments in the body, adapted for receiving liquid samples; wherein the device does not permit significant transfer of liquid sample either between the plurality of sample compartments or between any sample compartment and the surroundings.

In further embodiments of the foregoing multi-sample holding devices, the plurality of sample compartments comprises at least 2 sample compartments, at least 10 sample compartments, at least 16 sample compartments, at least 25 sample compartments, at least 36 sample compartments, at least 48 sample compartments, at least 72 sample compartments, or at least 96 sample compartments. In another embodiment of the foregoing multi-sample holding devices, the plurality of sample compartments comprises at least 96 sample compartments. In another embodiment of the foregoing multi-sample holding devices, the plurality of sample compartments comprises 96 sample compartments.

In one embodiment of the multi-sample holding devices, the sample compartments have openings on the top side of the device, and the openings of the sample compartments are sealed by placing a sealing mat on top of the device so as to cover the openings of the sample compartments. The sealing mat can be maintained in place by a constant-tension clamp. In another embodiment of the multi-sample holding device, the sample compartments are sealed by placing heat-sealed septums in the openings of the compartments prior to loading the compartments with samples. The samples can be loaded via needle injection through the septums. An adhesive polymeric membrane can then be placed on top of the device and septums to ensure adequate sealing.

In further embodiments of the foregoing multi-sample holding devices, the body of the devices is formed from a material selected from the group consisting of polyethyleneterephthalate, high-density polyethylene, polystyrene, and polystyrene-butadiene block copolymers. In another embodiment of the foregoing multi-sample holding devices, the body is formed from polyethylene-terephthalate. In another embodiment of the foregoing multi-sample holding devices, the body is formed from polystyrene-butadiene block copolymers.

In another embodiment, the invention embraces a container for pressure treatment of a liquid sample, where the container comprises at least one compartment for holding the liquid sample, where the container is fabricated from a flexible material, where the material can withstand up to about 5 kbar, preferably up to about 10 kbar of pressure without breakage or rupture and optionally is substantially impermeable to oxygen at high pressure. (The pressure indicated is a multidimensional pressure on the entire container, not a differential pressure.) In one embodiment, the container has only one compartment for holding the liquid sample. In one embodiment, the container has a constant loading volume at standard pressure. In another embodiment, the container has a variable loading volume at standard pressure.

In another embodiment, the container having a variable loading volume comprises a cylinder having a first end and a second end. A moveable plug is inserted into the first end of the cylinder; and a removable portion is affixed to the second end of the cylinder which can be detached to allow removal of the contents of the cylinder. The removable portion can be a cap, which can be threaded and engage with complementary threads on the second end of the cylinder, or can snap on, or can be affixed magnetically. In another embodiment, a short narrow protrusion extends from the second end of the cylinder, bearing threads or other methods of engaging a cap; the cap is placed on the protrusion, for later removal to allow removal of the contents of the cylinder. In one embodiment, the narrow protrusion can bear Luer-Lok® fittings (Luer-Lok® is a registered trademark of Becton, Dickinson & Co., Franklin Lakes, N.J. for an interlocking connection system).

In another embodiment, the container having a variable loading volume comprises a cylinder having a first end and a second end. A moveable plug inserted into the first end of the cylinder. A sealed tip is attached to the second end of the cylinder which can be detached to allow removal of the contents of the cylinder. The sealed tip can be a short narrow protrusion from the second end of the cylinder which can be broken off to allow removal of the contents of the cylinder. In some embodiments, the tip can be broken off manually; in other embodiments, the tip cannot be broken off manually and is broken off using a cutting tool.

In another embodiment, the moveable plug for use in the variable volume loading container has a one-way valve. The one-way valve plug allows air and sample within the container to be bled out at standard pressure, while preventing flow back through the valve into the container of any air, gas, or liquid from outside the container. In one embodiment, the one-way valve is a check valve. In another embodiment, the one-way valve is a ball check valve. In another embodiment, the one-way valve is a ball-and-spring check valve. In another embodiment, the one-way valve is a flap check valve. In another embodiment, the one-way valve is a duck bill backflow valve. In another embodiment, the one-way valve is an umbrella valve. In another embodiment, the one-way valve is a swing-check valve. In another embodiment, the one-way valve is a lift-check valve.

In further embodiments of the foregoing containers, the container is formed from a material selected from the group consisting of polyethyleneterephthalate, high-density polyethylene, polystyrene, and polystyrene-butadiene block copolymers. In another embodiment of the foregoing containers, the container is formed from polyethylene-terephthalate. In another embodiment of the foregoing containers, the container is formed from polystyrene-butadiene block copolymers.

In another embodiment, the invention embraces a system for solution exchange (solution mixing) under pressure, comprising a first container holding a first liquid sample and one or more additional containers holding an additional liquid sample or samples, where the first liquid sample and additional liquid sample or samples can be the same or different, where the containers are fabricated from materials that can withstand up to about 5 kbar, preferably up to about 10 kbar of pressure (multidimensional pressure on the system, not a differential pressure) without breakage or rupture and optionally are substantially impermeable to oxygen at high pressure, and where the liquid sample of the one or more additional containers can be mixed with the liquid sample of the first container while both first and additional containers and their respective liquid samples can be maintained at high pressure before, during, and after mixing. When the one or more additional containers comprise a plurality of containers, i.e., two or more additional containers, the contents of the two or more additional containers can be mixed with the contents of the first container either independently of the other two or more additional containers (i.e., at different times), or in conjunction with the other two or more additional containers (i.e., simultaneously or in a pre-determined time series). The high pressure before mixing or contacting, the high pressure during mixing or contacting, and the high pressure after mixing or contacting can all be the same pressure, or two can be the same pressure and one can be different pressures, or all three pressures can be different pressures.

In another embodiment of the system for solution exchange (solution mixing) under pressure, the system comprises at least two pre-mix containers holding liquid samples (where the liquid samples can be the same or different) which are designated the pre-mix containers, and at least one additional container designated the receiving container, where the receiving container can be empty prior to transfer or can contain a liquid or solid composition prior to transfer, where the containers are fabricated from materials that can withstand up to about 5 kbar, preferably up to about 10 kbar of pressure (multidimensional pressure on the system, not a differential pressure) without breakage or rupture and optionally are substantially impermeable to oxygen at high pressure, where the liquid samples in the at least two pre-mix containers holding liquid samples can be moved into the at least one receiving container where the liquid samples can contact each other, and the at least two pre-mix containers holding liquid samples, the at least one receiving container, and the liquid samples themselves can be maintained at high pressure before, during, and after mixing. In one embodiment, a mixing device, such as a static mixer (such as those used for HPLC solvent mixing), can be interposed in the fluid path between the at least two pre-mix containers holding liquid samples and the at least one receiving container in order to facilitate mixing of the liquid samples. In other embodiments, flow from one or more of the pre-mix containers can be controlled independently by valves, to allow contents from certain pre-mix containers to be drawn into the receiving container, while preventing flow from other selected pre-mix containers; at a later period, the valves can be set to permit the contents of the other selected pre-mix containers to flow into the receiving container.

In another embodiment of the system for solution exchange (solution mixing), the invention comprises a first container, where the first container comprises a compartment for holding a first liquid sample, the first container is fabricated from a flexible material that can withstand up to about 5 kbar, preferably up to about 10 kbar of pressure (multidimensional pressure on the system, not a differential pressure) without breakage or rupture and optionally is substantially impermeable to oxygen at high pressure; and one or more additional containers, where the one or more additional containers are fabricated from a flexible material that can withstand up to about 5 kbar, preferably up to about 10 kbar of pressure (multidimensional pressure on the system, not a differential pressure) without breakage or rupture and optionally is substantially impermeable to oxygen at high pressure, and where the one or more additional containers are completely enclosed by the first container, and where the one or more additional containers contains additional liquid samples, which can be the same or different from each other and from the first liquid sample; where the one or more additional containers can be opened while within the first container (either independently of the other additional containers, or in concert with the additional containers), allowing the first liquid sample and additional liquid samples to mix. In one embodiment, the one or more additional containers comprise a cap which can be maintained in a closed position, where the cap can be opened without opening the first container, and while the first container, one or more additional containers, and all liquid samples can be maintained at high pressure before, during, and after mixing. In another embodiment, the cap is also capable of mixing the liquid sample contained in the first container with the liquid samples of the one or more additional containers. In another embodiment, the cap comprises a magnetized portion, such as a magnetic disk.

In another embodiment of the system for solution exchange (solution mixing), the invention embraces a container system for pressure treatment of a liquid sample, comprising a first container which comprises a compartment for holding the liquid sample, where the first container is fabricated from a flexible material, where the material can withstand up to about 5 kbar, preferably up to about 10 kbar of pressure (multidimensional pressure on the system, not a differential pressure) without breakage or rupture and optionally is substantially impermeable to oxygen at high pressure; and also comprising at least one additional container, where the at least one additional container is fabricated from a flexible material, where the material can withstand up to about 5 kbar, preferably up to about 10 kbar of pressure (multidimensional pressure on the system, not a differential pressure) without breakage or rupture and optionally is substantially impermeable to oxygen at high pressure, and where the first container and the at least one additional container are connected by a flow loop. The flow loop comprises a check valve which permits flow in the flow loop in only one direction, and a pump capable of operating when the container system is subjected to high pressure. The pump can be controlled by a microprocessor. When the microprocessor is included within the high pressure apparatus, it can be powered by a battery which is also included within the high pressure apparatus, or by power lines which are run into the high pressure apparatus. Alternatively, the device can be controlled by drive shafts which enter the high pressure chamber through appropriately sealed openings into the high pressure chamber. The flow loop can bypass one or more of the one or more additional containers via bypass shunts; valves can close the bypass shunts and connect the one or more additional containers to the flow loop, either independently of other additional containers, or in concert.

In all of the embodiments for solution exchange (solution mixing), the liquid sample or samples in the at least one additional container, when mixed with the liquid sample in the first container, can alter the solution conditions of the first liquid sample in the first container, so that the combined liquid is at a different solution condition that the first liquid sample and/or the at least one additional liquid samples. The solution conditions that can be changed include, but are not limited to, pH, salt concentration, reducing agent concentration, oxidizing agent concentration, both reducing agent concentration and oxidizing agent concentration, chaotrope concentration, arginine concentration, surfactant concentration, preferentially excluding compound concentration, ligand concentration, concentration of any compounds originally present in the solution, or addition of another reactant or reagent.

In all of the foregoing embodiments of the devices, the device can comprise a material that permits a change in oxygen concentration due to oxygen mass transfer across the material of no more than about 0.2 mM in the sample during the duration of the high pressure treatment. In another embodiment, the material permits a change in oxygen concentration due to oxygen mass transfer across the material of no more than about 0.1 mM in the sample during the duration of the high pressure treatment. In another embodiment, the material permits a change in oxygen concentration due to oxygen mass transfer across the material of no more than about 0.05 mM in the sample during the duration of the high pressure treatment. In another embodiment, the material permits a change in oxygen concentration due to oxygen mass transfer across the material of no more than about 0.025 mM in the sample during the duration of the high pressure treatment. In another embodiment, the material permits a change in oxygen concentration due to oxygen mass transfer across the material of no more than about 0.01 mM in the sample during the duration of the high pressure treatment. In another embodiment, the material permits a change in oxygen concentration in the sample due to oxygen mass transfer across the material of no more than about 10% of the initial oxygen content of the sample during the duration of the high pressure treatment. In another embodiment, the material permits a change in oxygen concentration in the sample due to oxygen mass transfer across the material of no more than about 5% of the initial oxygen content of the sample during the duration of the high pressure treatment. In another embodiment, the material permits a change in oxygen concentration in the sample due to oxygen mass transfer across the material of no more than about 2.5% of the initial oxygen content of the sample during the duration of the high pressure treatment. In another embodiment, the material permits a change in oxygen concentration in the sample due to oxygen mass transfer across the material of no more than about 1% of the initial oxygen content of the sample during the duration of the high pressure treatment. In the foregoing embodiments, the high pressure treatment can last for about 6 hours, about 12 hours, about 18 hours, about 24 hours, about 30 hours, about 36 hours, about 42 hours, or about 48 hours.

In another embodiment, the invention embraces methods of altering solution conditions under high pressure, comprising the steps of: providing at least one composition in a solution in a first container; providing at least one agent for changing solution conditions in at least one additional container, where the contents of the at least one additional container are not in contact with the contents of the first container; placing the containers under high pressure; and causing the contents of the at least one additional container to contact the contents of the first container. In another embodiment, the contents of the first and at least one additional container are mixed by convection. In another embodiment, the contents of the first and at least one additional container are mixed by agitation. In another embodiment, the contents of the first and at least one additional container are mixed by diffusion. In another embodiment, the contents of the first and at least one additional container are mixed by passing the contents through a mixer, such as a static mixer. In another embodiment, the contents of the first container and the at least one additional container are transferred to a receiving container, where the receiving container may be empty prior to transfer or may contain a liquid or solid composition prior to transfer; the contents of the first container and the at least one additional container can be mixed during or after the transfer to the receiving container. In another embodiment, the at least one additional container is contained within said first container. In another embodiment, the at least one additional container is in a flow path with the first container.

In one embodiment, the invention embraces methods of altering solution conditions under high pressure, comprising the steps of: providing at least one composition in a solution in a first container; providing at least one agent for changing solution conditions in at least one additional container, where the contents of the at least one additional container are not in contact with the contents of the first container; placing the containers under high pressure; and causing the contents of the at least one additional container to contact the contents of the first container, wherein the contents of the at least one additional container are caused to contact the contents of the first container over a period of time. In one embodiment, the contents of the at least one additional container are caused to contact the contents of the first container in a continuous manner, whereby the solution conditions of the contents of the first container are changed continuously over a period of time. In another embodiment, the contents of the at least one additional container are caused to contact the contents of the first container in a step-wise (discontinuous) manner (e.g., by mixing portions of solutions, waiting, and mixing additional portions of solutions), whereby the solution conditions of the contents of the first container are changed step-wise over a period of time. In one embodiment of this step-wise change in solution conditions, the pH of the contents of the first container is at about 9 to about 11, or at about 9.5 to about 10.5, or at about 10. In another embodiment of this step-wise change in solution conditions, the pH of the contents of the first container is at about 9 to about 11, or at about 9.5 to about 10.5, or at about 10, and is lowered to a pH of about 7 to about 8.9, or about 7.5 to about 8.5, or about 8. In another embodiment of the stepwise method, the pH is lowered by about 0.01 to about 2 pH units every approximately 24 hours, or by about 0.1 to about 1 pH unit every approximately 24 hours, or by about 0.1 to about 0.5 pH units every approximately 24 hours, or by about 0.1 to about 0.4 pH units every approximately 24 hours, or by about 0.1 to about 0.3 pH units every approximately 24 hours, or by about 0.2 pH units every approximately 24 hours.

In one embodiment of the method, the at least one composition in a solution in a first container is a protein. The protein can be in a non-native state, such as a denatured protein or an aggregated protein; the aggregated protein can be a soluble aggregate, insoluble aggregate, or inclusion body, or any mixture of the forgoing.

In one embodiment of the method, the at least one agent for changing solution conditions is an agent for changing the pH of the solution. In another embodiment, the at least one agent for changing solution conditions is an agent for changing the salt concentration of the solution. In another embodiment, the at least one agent for changing solution conditions is an agent for changing the reducing agent concentration, oxidizing agent concentration, or both reducing agent concentration and oxidizing agent concentration of the solution. In another embodiment, the at least one agent for changing solution conditions is an agent for changing the chaotrope concentration of the solution. In another embodiment, the at least one agent for changing solution conditions is an agent for changing the concentration of arginine of the solution. In another embodiment, the at least one agent for changing solution conditions is an agent for changing the concentration of surfactant of the solution. In another embodiment, the at least one agent for changing solution conditions is an agent for changing the preferentially excluding compound concentration of the solution. In another embodiment, the at least one agent for changing solution conditions is an agent for changing the ligand concentration of the solution. In another embodiment, the at least one agent for changing solution conditions is an agent for changing the concentration of any compounds originally present in the solution. In another embodiment, the at least one agent for changing solution conditions is an additional reactant or reagent to add to the solution.

In another embodiment, the containers are placed under at least about 250 bar of pressure. In another embodiment, the containers are placed under at least about 400 bar of pressure. In another embodiment, the containers are placed under at least about 500 bar of pressure. In another embodiment, the containers are placed under at least about 1000 bar of pressure. In another embodiment, the containers are placed under at least about 2000 bar of pressure. In another embodiment, the containers are placed under at least about 2500 bar of pressure. In another embodiment, the containers are placed under at least about 3000 bar of pressure. In another embodiment, the containers are placed under at least about 4000 bar of pressure. In another embodiment, the containers are placed under at least about 5000 bar of pressure. In another embodiment, the containers are placed under at least about 6000 bar of pressure. In another embodiment, the containers are placed under at least about 7000 bar of pressure. In another embodiment, the containers are placed under at least about 8000 bar of pressure. In another embodiment, the containers are placed under at least about 9000 bar of pressure. In another embodiment, the containers are placed under at least about 10,000 bar of pressure.

### BRIEF DESCRIPTION OF THE DRAWINGS

FIG. 1 depicts a top view of one embodiment of the invention, the multi-well plate design.

FIG. 2A depicts a side view of one possible embodiment of the multi-well design. The tops of the wells are partially covered in a “dome” to ensure venting of all air.

FIG. 2B depicts a side view of the “dome” covering the wells in FIG. 2A.

FIG. 3 depicts an example of the 96-well plate embodiment with sealing mat and clamp assembly that can be used to seal the “dome” inlets of FIG. 2A and FIG. 2B.

FIG. 4 depicts another embodiment of the invention, where heat-sealed septums are used to seal the wells of a multi-well embodiment of the invention.

FIG. 5 depicts another embodiment of the invention, the constant loading volume container.

FIG. 6 depicts another embodiment of the invention, the variable loading volume container.

FIG. 7A depicts a sectional view of a one-way valve assembly for use in the variable loading volume container. FIG. 7B depicts the one-way valve assembly as installed in the variable loading volume container.

FIG. 8 depicts an embodiment of the invention useful for mixing solutions at high pressure. In FIG. 8A, the secondary container is depicted in closed position. In FIG. 8B, the secondary container is depicted in open position.

FIG. 9 depicts another embodiment of the invention useful for mixing solutions at high pressure.

FIG. 10 depicts results of an experiment demonstrating oxygen transfer through materials which are not substantially oxygen-impermeable at high pressure. The effects of storage and pressurization conditions on oxygen transfer and GSH concentration are shown; the solutions conditions were pH 8.0, 4 mM GSH, 2 mM GSSG, 500 ml solution, 25° C., for 17 hours.

FIG. 11 depicts the calculated transfer of oxygen through the walls of a syringe made from various polymers (LDPE, low density polyethylene, top curve; HDPE, high density polyethylene, second curve from top; PS, polystyrene, third curve from top and second curve from bottom; PET, polyethylene-terephthalate, bottom curve), as a function of the oxygen concentration of the surroundings. Oxygen transfer is calculated for syringe walls as a function of polymer type, assuming 24 hours for transfer, 1/16 inch thickness, 1.5 inches length, and 0.25 inch outer diameter.

FIG. 12 depicts the amount of oxygen loaded in a sample containing an air bubble as a function of the bubble size in the sample, where the bubble size is calculated as the volume percent of the sample. The curve assumes PV=nRT, which is a suitable approximation for this calculation.

FIG. 13 depicts an overall view of an embodiment of a solution exchange device.

FIG. 14 depicts a cross-section of the solution exchange device of FIG. 13.

FIG. 15 depicts the pressure chamber portion of the solution exchange device of FIG. 13 and FIG. 14, prior to mixing of solutions.

FIG. 16 depicts the pressure chamber portion of the solution exchange device of FIG. 13 and FIG. 14, subsequent to mixing of solutions.

FIG. 17 depicts one of the pre-mixing containers of the solution exchange device of FIG. 13 and FIG. 14.

FIG. 18 depicts a receiving container of the solution exchange device of FIG. 13 and FIG. 14.

FIG. 19 depicts a check valve adapter useful in one of the variable loading volume embodiments of the invention.

FIG. 20 depicts a check valve useful in various embodiments of the invention, such as the check valve adapter depicted in FIG. 19. The arrow indicates the direction of permitted fluid flow.

FIG. 20A depicts the check valve of FIG. 20 installed in the check valve adapter of FIG. 19.

FIG. 21 depicts a variable loading volume embodiment of the invention, with the check valve adapter (with check valve installed, as depicted in FIG. 20A) inserted into the device to contain the liquid therein.

FIG. 22 depicts an experiment performing Coomassie Blue solution exchange under pressure. The open squares represent the actual sample (upper right square lying on solid line corresponds to initial conditions; lower right square with error bars corresponds to conditions after solution exchange). The solid line represents the calibration line from known concentrations of dye.

FIG. 23 depicts percent recovered lysozyme as a function of solution conditions. From left to right: 1M GdnHCl pressure treated aggregate, with no solution exchange; 0.5M GdnHCl pressure treated aggregate, with no solution exchange; aggregate solution exchanged under pressure from 1 to 0.5M GdnHCl; 1M GdnHCl atmospheric pressure control aggregate, no solution exchange; 0.5M GdnHCl atmospheric pressure aggregate, no solution exchange; and aggregate solution exchanged under atmospheric pressure from 1 to 0.5M GdnHCl, respectively. All samples were placed in a refolding buffer of 50 mM Tris-HCl, 5 mM GSSG, 2 mM DTT, pH 8.0 at 25° C.

### DETAILED DESCRIPTION OF THE INVENTION

By “high pressure” is meant a pressure of at least about 250 bar. The pressure at which the devices of the invention are used can be at least about 250 bar of pressure, at least about 400 bar of pressure, at least about 500 bar of pressure, at least about 1 kbar of pressure, at least about 2 kbar of pressure, at least about 3 kbar of pressure, at least about 5 kbar of pressure, at least about 6 kbar of pressure, at least about 7 kbar of pressure, at least about 8 kbar of pressure, at least about 9 kbar of pressure, or at least about 10 kbar of pressure.

By “closed system” is meant the standard chemical thermodynamic term referring to a system where matter cannot be transferred between the system and its surroundings; however, transfer of mechanical or heat energy can occur between a closed system and its surroundings. In contrast, an “open system” permits transfer of matter and/or mechanical or heat energy between the system and its surroundings. An “isolated system” is a closed system that does not permit either mechanical or thermal contact with its surroundings, i.e., no transfer of mechanical or heat energy takes place to or from an isolated system. A “substantially closed system” is a system where less than about 1%, more preferably less than about 0.5%, more preferably less than about 0.2%, more preferably less than about 0.1%, more preferably less than about 0.05%, still more preferably less than about 0.01% of the mass of the sample can be transferred between the system and its surroundings.

By “significant transfer of liquid sample” is meant a transfer of about 1% or more of the volume of liquid contained in a sample (measured at standard atmospheric pressure). When devices of the invention are designed to prevent significant transfer of liquid sample, the amount of sample transferred during the use of the device is less than about 1%, more preferably less than about 0.5%, more preferably less than about 0.2%, more preferably less than about 0.1%, more preferably less than about 0.05%, still more preferably less than about 0.01% of the unpressurized volume of the sample.

By “substantially impermeable to oxygen at high pressure,” “substantially oxygen-impermeable at high pressure,” or “substantially impermeable to oxygen mass transfer at high pressure” is meant a material that permits a change in oxygen concentration due to oxygen mass transfer across the material of no more than about 0.3 mM in the sample during the duration of the high pressure treatment. In another embodiment, the material permits a change in oxygen concentration due to oxygen mass transfer across the material of not more than about 0.2 mM, preferably not more than about 0.1 mM, preferably not more than about 0.05 mM, more preferably not more than about 0.025 mM, still more preferably not more than about 0.01 mM, in the sample during the duration of the high pressure treatment. In percentage terms, the material permits a change in oxygen concentration in the sample due to oxygen mass transfer across the material of no more than about 10%, preferably no more than about 5%, more preferably no more than about 2.5%, still more preferably no more than about 1%, of the initial oxygen content of the sample during the duration of the high pressure treatment.

### Materials for High-Pressure Devices

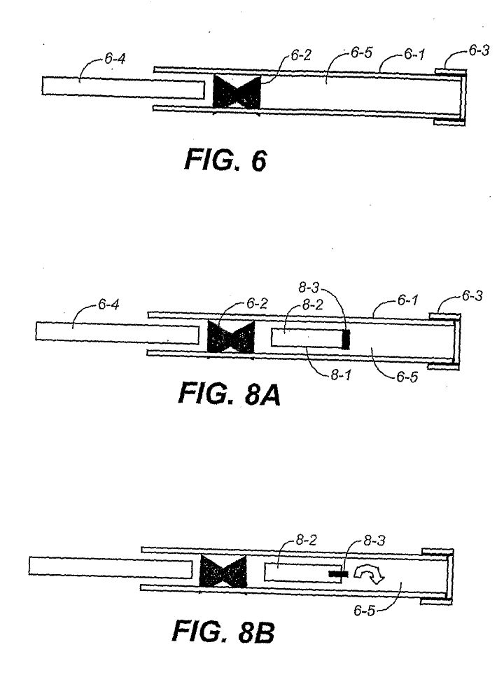

The body of the high-pressure devices can be fabricated from a wide variety of materials. If a device does not have at least one moveable surface which can transmit pressure (such as the variable loading volume device depicted in FIG. 6), then the materials from which the device is made should be flexible to enable pressure transfer. Suitable materials should not break, fracture, or otherwise undergo any failure or loss of integrity under high pressure treatment which would permit leakage of samples either from one or more sample compartments to the external surroundings, or allow leakage of fluids, gases, or other materials in the external surroundings of the container into the one or more sample compartments, or which would permit leakage of samples between sample compartments. Such leakage, of course, is not meant to include intentional transfers between one or more sample compartments and the external surroundings, or intentional transfers between two or more sample compartments or other compartments, which are deliberately desired by the artisan.

The device must be constructed with a material that can withstand at least about 250 bar of pressure and still maintain integrity. In another embodiment, the material can withstand at least about 500 bar of pressure and still maintain integrity. In another embodiment, the material can withstand at least about 1 kbar of pressure and still maintain integrity. In another embodiment, the material can withstand at least about 2 kbar of pressure and still maintain integrity. In another embodiment, the material can withstand at least about 3 kbar of pressure and still maintain integrity. In another embodiment, the material can withstand up to about 5 kbar, preferably up to about 10 kbar of pressure, and still maintain integrity. The specified pressures are multi-dimensional pressure on the device, not a pressure differential or pressure drop across the device. That is, the container or containers used as the device are placed in a pressure chamber which is pressurized to the specified pressure; while the pressure chamber must be capable of withstanding a pressure differential or pressure drop of up to 5 kbar or 10 kbar within the chamber versus atmospheric pressure outside of the chamber, the material of the container does not experience such a dramatic differential pressure, but rather a uniform pressure from all directions.

The material used should also permit pressure transfer from the surroundings to the sample compartments, so that the pressure across the device is roughly equivalent; that is, the difference in pressure experienced by any two locations within the device is no more than about 1%, preferably no more than about 0.5%, more preferably no more than about 0.1%, of the total pressure. In other embodiments, the absolute difference in pressure experienced by any two locations within the device is less than about 5 bar, preferably less than about 2 bar, more preferably less than about 1 bar. Any difference between the external applied pressure and any interior portion of the device is no more than about 5%, preferably no more than about 2%, more preferably no more than about 1%, still more preferably no more than about 0.5%, yet more preferably no more than about 0.1%, of the total applied external pressure. Therefore, the material should be flexible in order to transmit pressure.

In one embodiment, the materials are polymers. In another embodiment, the polymeric materials can be injection molded for inexpensive mass production. Suitable polymeric materials include polyethyleneterephthalate, high-density polyethylene, polystyrene, and polystyrene-butadiene block copolymers. Other polymeric materials which can withstand high-pressure treatment, but which are not necessarily oxygen impermeable, include low-density polyethylene, polypropylene, and polycarbonate.

### Considerations of Oxygen Content of Sample

Many reactions, such as refolding of cysteine-containing proteins, can be affected by the oxygen content of the sample. Typically a protein refolding experiment will entail use of a specified concentration of redox reagents such as thiols (e.g., glutathione, cystamine, cystine, dithiothreitol, dithioerythritol; in reduced form, oxidized form, or a mixture of reduced and oxidized forms for, e.g., disulfide shuffling). The concentration of oxygen in a sample can be affected by the presence of air bubbles in a sample, as air bubbles will be forced into solution at high pressures, changing the O₂ concentration in the sample. The concentration of oxygen in a sample can also be affected by diffusion of oxygen across the walls of the device. The sample device will typically be placed in a chamber to which pressure is applied; if the fluid used in the chamber is water, then oxygen dissolved in the chamber's water surrounding the sample device can diffuse across the walls of the device.

These considerations are addressed in the following sections, “oxygen concentration changes due to air bubbles,” and “oxygen permeability at high pressure.”

### Oxygen Concentration Changes Due to Air Bubbles

It is estimated that about 80% of the variation in oxygen concentration will arise from air bubbles in the sample, while about 20% of the variation will arise from oxygen diffusion across the walls of a syringe-type device (where the device is not substantially impermeable to oxygen transfer at high pressure). This underscores the importance of removing as many air bubbles as possible from the sample vial. For every 25 μl of air in a 1 ml sample, 0.2 mmoles O₂ is loaded, as high pressure will dissolve the air into the liquid sample; that amount of oxygen will react with 0.8 mM reduced thiol. Typical reduced thiol concentrations range from about 1 mM to about 10 mM (Clark E. D., “Protein refolding for industrial processes,” Curr. Opin. Biotechnol. 12:202-207 (2001)), and over this range a 0.8 mM change in reduced thiol concentration will cause a variation in concentration of from about 8% to about 80%. At a typical concentration of 4 mM reduced thiol, a 0.8 mM reduction of reduced thiol results in about a 20% change in solution concentration of reduced thiols. This underscores the importance of removing all air bubbles, which is difficult to accomplish with current state-of-the-art vials and which the instant invention is designed to address.

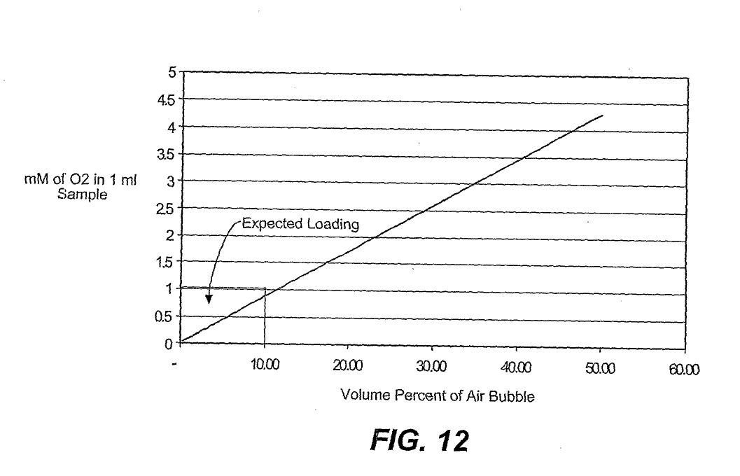

FIG. 12 shows the oxygen loading caused by air bubbles of various sizes. The volume percent of air bubbles should be kept as low as possible, to no more than about 10% of the sample volume, more preferably no more than about 5% of the sample volume, still more preferably no more than about 2.5% of the sample volume, yet more preferably no more than about 1% of the sample volume.

### Oxygen Permeability at High Pressure

The materials used in the devices are optionally substantially impermeable to oxygen mass transfer at high pressure. Materials which are substantially impermeable to oxygen should be used when oxygen transfer may affect the sample being studied or treated using the device. Optionally, the materials used are also substantially impermeable to transfer of other gases at high pressure, such as carbon dioxide, which may affect the sample being studied or treated using the device. Materials which are substantially impermeable to oxygen mass transfer at high pressure include, but are not limited to, polyethylene-terephthalate (PET or PETE), Mylar® (Mylar is a registered trademark of DuPont, designating a biaxially-oriented polyethylene terephthalate polyester film), high-density polyethylene, and polystyrene. Alternatively, if vessels walls are made thick enough, materials which are less impermeable to oxygen can be used. Finally, materials which are more permeable to oxygen, including, but not limited to, polystyrene-butadiene block copolymers such as Styrolux® (e.g., Styrolux® 684D) can be used with suitable coatings of other polymers or other materials to decrease their oxygen permeability. (Styrolux® is a registered trademark of BASF Aktiengesellschaft Corp., Ludwigshafen, Germany, and Westlake Plastics Company, Lenni, Pa., for styrene resins.)

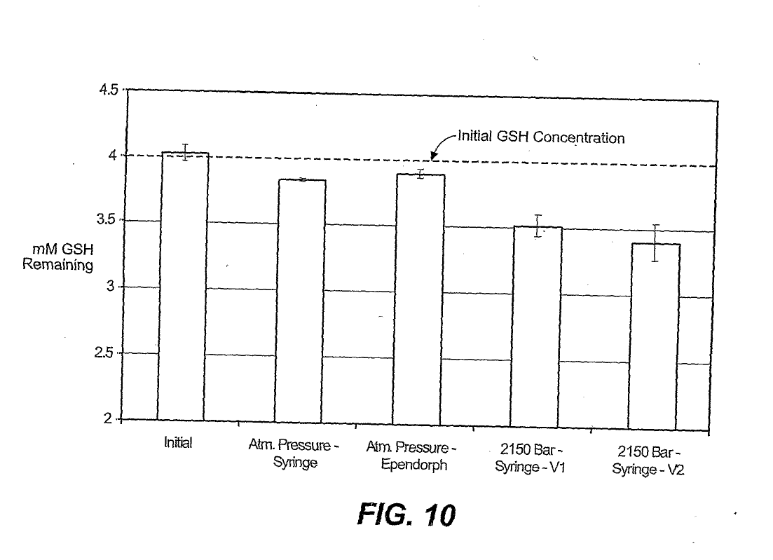

Experimental evidence confirms the utility of using substantially oxygen-impermeable materials at high pressure when oxygen affects the sample being studied or treated using the device. Up to about 0.35 micromoles of O₂ can be transferred during a typical pressure experiment, enough to significantly alter the redox environment of a solution. FIG. 10 depicts an experiment done with conventional syringes currently used for high-pressure treatment. The syringes used were 1 ml low-density polyethylene syringes from Becton Dickinson. A 500 ml aqueous solution at pH 8.0, 4 mM GSH (reduced glutathione), 2 mM GSSG (oxidized glutathione), was kept at 2150 bar for 17 hours. As indicated in FIG. 10, enough oxygen was transferred to lower the concentration of reduced glutathione from 4.0 mM to 3.5 mM or less.

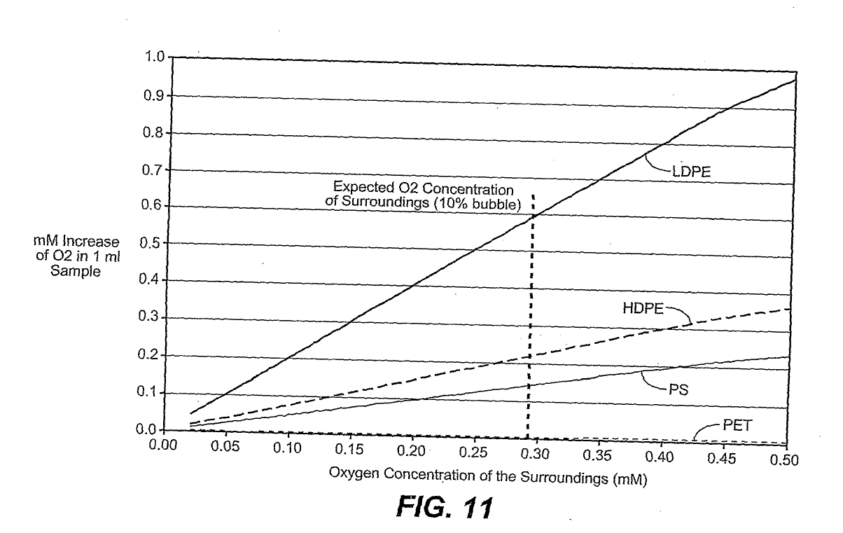

FIG. 11 depicts calculations of the amount of oxygen transfer across the walls of a syringe used under high pressure. The calculations are for a syringe of 1/16 inch thickness, 1.5 inch length, and 0.25 inch outer diameter. The calculation assumed a 24 hour experiment at 2000 bar with a variable surrounding oxygen concentration; the expected oxygen concentration in the surrounding fluid will likely be about 0.3 mM (under the assumption that about 10% of the volume of the surroundings is made up of an air bubble before compression), and is indicated with a vertical dashed line in FIG. 11. The oxygen in the bubble is calculated by simply using the ideal gas law to calculate the amount of air in the bubble at standard temperature and pressure; at higher pressure, the air will dissolve into the solution. The calculation is performed using Fick's law of diffusion at steady-state; diffusion coefficients are used instead of permeability coefficients, as the solubility of O₂ in polymers increases dramatically at high pressures. The diffusion coefficients were taken from the Polymer Handbook, 4^{th} Edition; editors, J. Brandup, E. H. Immergut, and E. A. Grulke; associate editors, A. Abe, D. R. Bloch; New York: Wiley, 1999.

With these assumptions, the calculations indicate that, at the likely value of oxygen concentration in the surrounding liquid, approximately 0.2 mM equivalents of O₂ is transferred in tubes made of HDPE, and 0.6 mM equivalents of O₂ with LDPE. Oxygen transfer across a polypropylene device was not calculated, but based on relative permeability values, is believed to lie between the values for HDPE and LDPE. Polyethylene terephthalate (PET or PETE) is calculated to have almost no transfer of oxygen under the conditions assumed. Consequently, this calculation demonstrates that materials can be judiciously chosen to significantly reduce or almost eliminate oxygen transfer through the polymeric walls of the devices.

### Device Embodiments: Multi-Well Plates

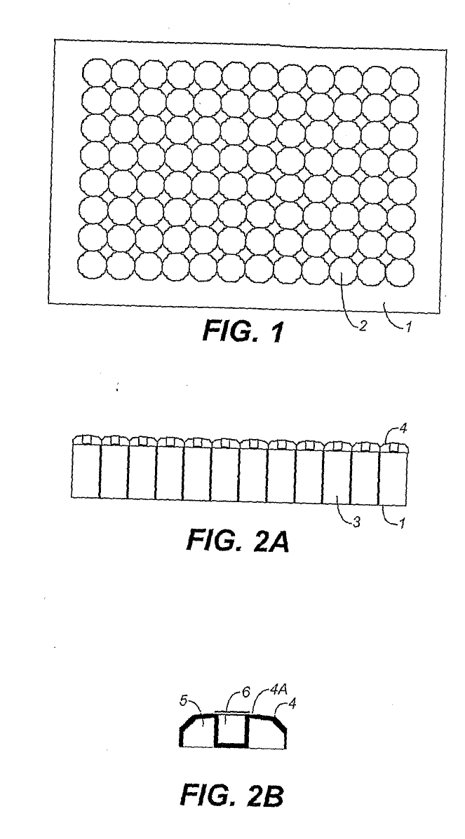

In one embodiment, the high-pressure device comprises a plurality of wells in a body or plate (“multi-well plate”). One example of such an embodiment is shown in FIG. 1. The embodiment shown is a 96-well plate; a body (1) made of a flexible material substantially impermeable to oxygen mass transfer at high pressure has ninety-six wells (2) for holding liquid samples. The material is preferably (but not necessarily) chosen so that the plate can be formed by injection molding.

Once a suitable material has been chosen for the body of the multi-well plate embodiment, the samples must be introduced into the sample compartments. The inclusion of air pockets in the sample wells is undesirable, as the oxygen in the air will be driven into solution under high pressure, altering the redox environment of the sample, and the presence of air pockets may also cause excessive strain on the material. The wells are thus designed so as to eliminate, to the greatest extent possible, any residual air left in the wells.

FIG. 2A depicts a side view of one possible embodiment of the well design (1). The wells (3) are partially covered in a “dome” (4) to ensure venting of all air. The dome (4) is shown in larger detail in FIG. 2B. The region (6) is the inlet for sample loading, and is surrounded by solid material (5) forming the dome. The inlet (6) should be large enough to enable insertion of the appropriate sized pipette tip for reagent delivery and the venting of air. This domed design enables overfilling to vent all air in the well prior to sealing. Additionally, during the overfill, excess sample will drain down the sides of the dome and will eliminate cross-contamination between samples. The dome (4) has a substantially flat surface on top, in an (4A) area closely surrounding the inlet, in order to provide an adequate sealing surface. The dimensions are selected to enable sample loading with standard-sized pipette tips, to enable sample venting, to have sufficient troughs at the base of the domes to prevent cross-contamination, and to provide the previously mentioned flat top to provide an adequate sealing surface.

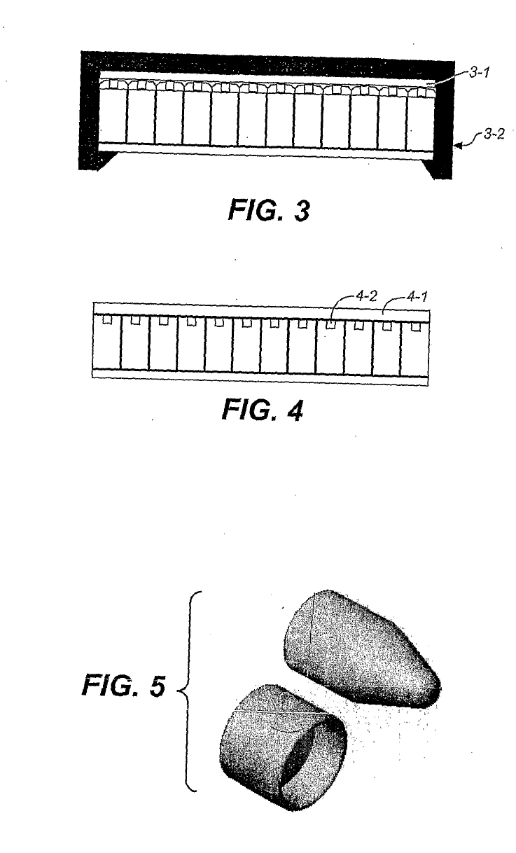

In one embodiment of the multi-well device, a mat is placed on the top of the multi-well plate to seal the wells. Materials suitable for such a mat include, but are not limited to, silicone rubber. In one embodiment, the mat has a thickness of approximately ⅛ inch, with length and width substantially identical to that of the multi-well plate it is to be used with. The mat should be made from a material of sufficient flexibility to enable a good seal on top of the domes, and to allow deformation due to pressure-induced volumetric changes within the sample. As there is no differential pressure across the sealing surface in this embodiment—that is, the pressure experienced by the sample inside the well is substantially similar to the pressure experienced by the mat—the sealing mat need not provide any additional sealing capacity than that expected at atmospheric pressure. If the sealing material used is not substantially impermeable to oxygen at high pressure, a film which is substantially impermeable to oxygen at high pressure can be placed on the sealing mat to inhibit oxygen transfer. The film can be made from materials including, but not limited to, Mylar®. In this embodiment of the multi-well device using a sealing mat, a clamp is affixed on the plate in order to place force on the sealing mat and enable sealing of the wells. The clamp should provide uniform force across the device, and sufficient force to ensure an adequate seal. The clamp should also provide constant force throughout the pressurization cycle, which requires a constant tension clamp (not a constant force clamp) due to the contraction of materials (especially the sealing mat) at high pressure. FIG. 3 depicts the 96-well plate embodiment with sealing mat (3-1) and clamp assembly (3-2).

In another embodiment of the multi-well plate, depicted in FIG. 4, the wells of the plate are not covered by a dome and sealing mat; instead, the wells are covered by heat-sealed septa (4-2) prior to loading the wells with samples. Such septums are commonly used when sealing medical vials. The heat sealed septum ensures a sealed well and prevents sample contamination. Samples are loaded into this embodiment of the multi-well plate by injection with a multi-channel pipetter equipped with needles rather than pipettes. The needles penetrate the septum in order to fill the sample wells. A secondary needle also pierces the sample concurrently with filling, in order to vent air and to allow the well to fill completely. Multi-channel pipetters are available commercially which are designed for pipetting solutions into multi-well plates; such a pipetter can be easily adapted to use a needle for sample loading instead of a pipette tip. After sample loading, the septum is covered with a secondary, adhesive polymeric membrane (4-1). The membrane seals the pierced holes created during sample loading. Optionally, the membrane can also inhibit oxygen diffusion across the septum; that is, the membrane can be substantially impermeable to oxygen. Potential materials for the adhesive polymeric membrane include, but are not limited to, Mylar®.

### Device Embodiments: Constant Loading Volume Devices

In another embodiment, the high-pressure device comprises a container where the volume of the container is fixed at standard pressure; this embodiment is designated the constant loading volume device. The entire container shrinks proportionally upon exposure to high pressure; typically, the container will shrink by about 5%-10% of its volume at 2 kbar, and by about 20% (estimated) of its volume at 4 kbar; hence, for fabricating this device, a flexible material should be used. One example of such an embodiment is shown in FIG. 5. This device consists of a cylindrical barrel which has a conical bottom. The container can be fabricated with a wide variety of internal volumes; examples of dimensions for containers having 250 μL, 500 μL, 750 μL, or 1000 μL are specified in Table 1. The interior of the container can be graduated, for example at 50 μL increments. The top of the cylindrical barrel can be threaded for seal with a screw cap (which can be shaped as the conical bottom in FIG. 5, or which can also be cylindrical). The threaded screw cap should be capable of maintaining a seal when there is at least about 5 psig pressure differential between the interior and exterior (note that the 5 psig is a differential pressure, not a total pressure; pressure differentials of this magnitude are similar to those of commercial bottles containing carbonated beverages). Typical sizes of embodiments of the constant loading volume device are given in Table 1 (the thickness of the walls of these particular embodiments of the constant loading volume device is 1/16 inch).

TABLE 1
Dimensions for Internal Volume specified

| Internal Volume | 250 μL | 500 μL | 750 μL | 1000 μL |
|---|---|---|---|---|
| Length (cm) | 0.94 | 0.90 | 1.07 | 1.21 |
| Inner Diameter (cm) | 0.5 | 0.720793 | 0.825102 | 0.908142 |
| Total Height (cm) | 2.44 | 2.40 | 2.57 | 2.71 |
| Cone Volume (mL) | 0.064795 | 0.134656 | 0.176449 | 0.213753 |
| (1 cm height) | | | | |

### Device Embodiments: Variable Loading Volume Devices

In another embodiment, the high-pressure device comprises a container of variable loading volume. An example of this embodiment is shown in FIG. 6. In this embodiment, a cylinder (6-1) with a moveable plug (6-2) and a removable cap (6-3) is provided, in a fashion similar to a syringe. In one embodiment, when the cylinder is vertically oriented, the plug forms the bottom of the sample compartment, and acts as a seal between the sample compartment and the external environment. The plug can have an attached plunger rod, or a separate plunger rod (6-4), which can be used to move the plug to the desired volume before filling the cylinder. The cylinder can be graduated, for example in 50 μL increments, in order to guide placement of the plug to the desired volume. The sample is contained in the interior space (6-5) of the device. The cylinder can then be filled with the desired sample, with care taken to remove as much residual air as possible. The removable cap is then placed on the top of the cylinder. In an alternative embodiment, the cylinder can be filled in the opposite orientation, i.e., the cylinder can be oriented so the cap is placed on the bottom of the cylinder, and the plug is inserted into the top of the cylinder. In one embodiment, the removable cap is threaded, and can simply be screwed on to the top of the cylinder. The threaded removable cap should be capable of maintaining a seal when there is at least about 5 psig pressure differential between the interior and exterior (note that the 5 psig is a differential pressure, not a total pressure; pressure differentials of this magnitude are similar to those of commercial bottles containing carbonated beverages). The sample can then be treated under pressure; after the pressure treatment, the cap can be removed and the contents of the cylinder are poured out, or pushed out by pushing the plug with the plunger rod.

In an alternative embodiment, the removable cap is replaced with a breakable tip on the closed end of the cylinder. In this embodiment, a liquid sample is placed in the cylinder and the plug is inserted at the top of the cylinder. The breakable tip is kept intact during the high-pressure treatment. After the treatment, the tip is broken off, and the contents of the cylinder are poured out, or pushed out by pushing the moveable plug with the plunger rod. The tip can be designed to be broken off by hand, or can be designed to be broken off by a cutting tool; see FIG. 21 for an example of a variable loading volume device where the tip can be removed by a cutting tool in order to expel the sample.

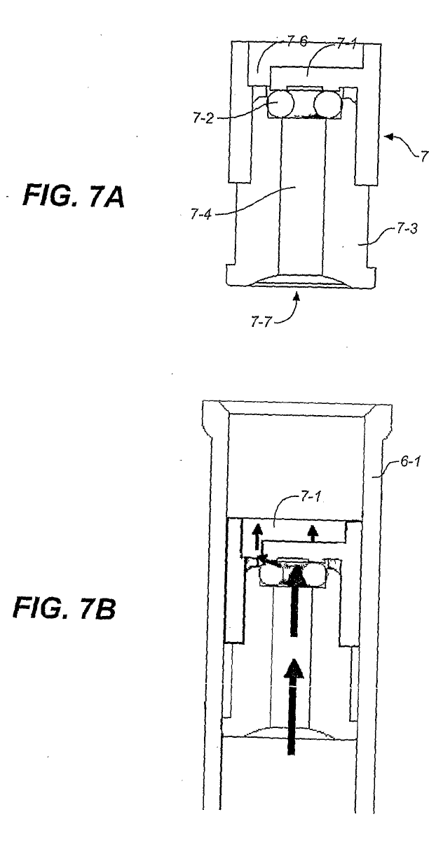

In another alternative embodiment, a needle can be run between the moveable plug and the cylinder wall to insert the sample into the cylinder. A one-way valve on the moveable plug allows expulsion of air in the cylinder as the sample is introduced (see FIG. 7A, FIG. 7B, and discussion of a one-way valve assembly below).

It should be noted that, as the moveable plug will move in response to applied pressure, the material used to fabricate the variable loading volume device need not be as flexible as the material used in the other devices of the invention.

In FIG. 7A and FIG. 7B, a moveable plug (7) is shown which is particularly adapted for high-pressure applications, and which can function as a one-way valve plug. The embodiment shown in FIG. 7A and FIG. 7B is a flap plug or flap valve, as it relies on a flap to allow one-way flow of liquid. In FIG. 7A flexible flap (7-1) forms a seal with the O-ring (7-2). The flap (7-1) and O-ring (7-2) seal the external environment (7-6) from the internal passage (7-4), which opens to the interior of the variable loading volume device at opening (7-7). Area (7-3) is solid. When the one-way valve plug is inserted into the polymeric barrel (6-1) of the variable loading volume device as depicted in FIG. 7B, the plug can be pressed down until sample begins to bleed out of the one-way valve. This occurs as the flexible flap (molded flap) bends to allow sample to escape via the path indicated by the arrows in FIG. 7B. Pressing down until sample bleeds out of the valve ensures exclusion of as much air as possible from the sample, and also allows adjustment of the amount of sample in the device. As the flap can bend only in one direction (away from the O-ring), neither air nor any other substance present external to the sample can flow back into the sample.

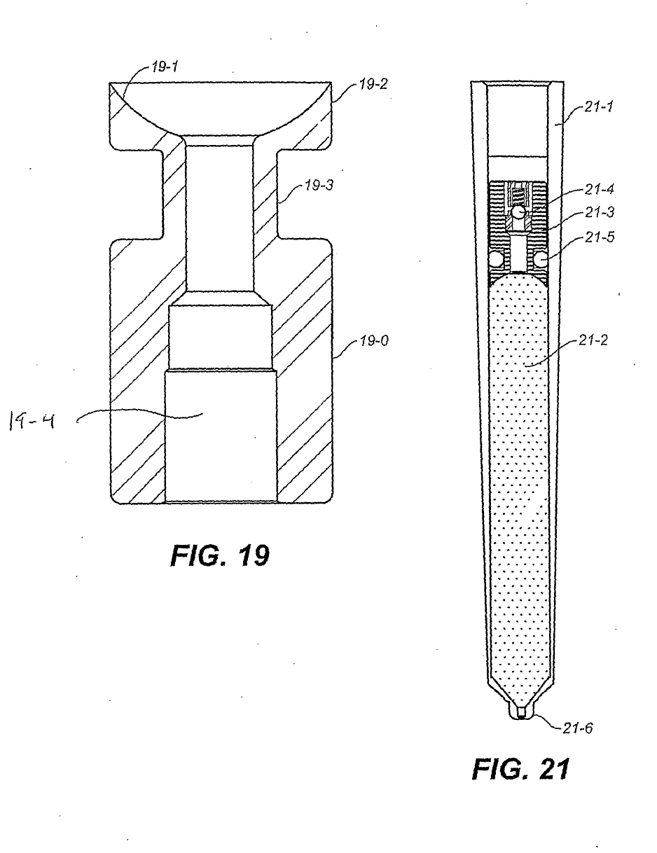

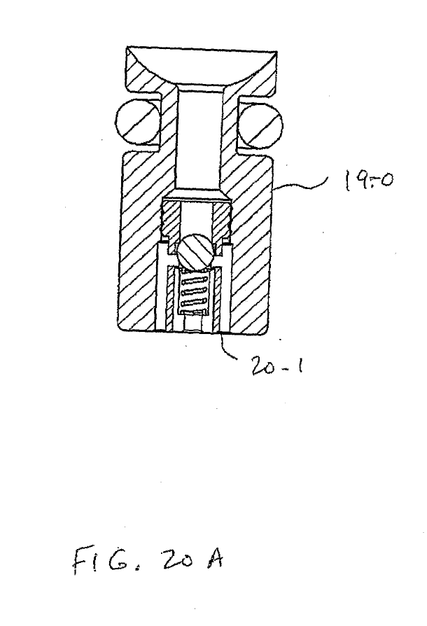

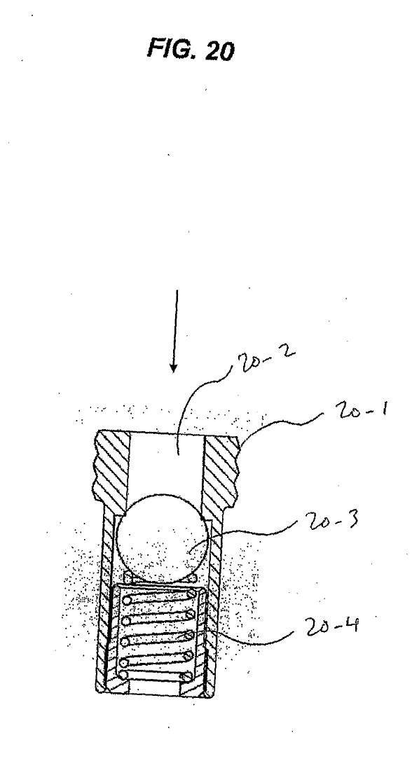

Another variable loading volume device suitable for use as a high pressure sample vial is shown in FIG. 21, comprised of a polymeric sample barrel, a check valve adaptor, a check valve assembly, and an O-ring seal. In FIG. 19, another design for a moveable plug (19-0) is shown which is particularly useful for high-pressure applications, and which can function as a one-way valve plug; this moveable plug also functions as a check valve adapter (that is, a check valve can be inserted into the moveable plug (19-0)). The plug can be manufactured from a variety of materials, including, but not limited to, Delrin® (Delrin® is a registered trademark of E. I. Du Pont de Nemours and Company, Wilmington, Del., for acetal resin); the plug can be fabricated by injection molding or by machining. The area of the plug that contacts the liquid sample is curved (19-1) (note that the plug would be inverted from the orientation shown in FIG. 19 when inserted into a container holding a liquid); this curvature ensures that as much air as possible is forced out of the container. Indentation (19-3) allows installation of an O-ring to form a moveable seal with the walls of the container. This plug is adapted to receive a check valve in its interior lumen (19-4), which can be easily installed by manually inserting the check valve into the adapter. The valve plug/check valve adapter preferably utilizes a ball-and-spring check valve. FIG. 20 depicts such a check valve (20-1), which is commercially available (The Lee Co, PN#CCPX0003349S A, Westbrook, Conn.). The arrow in FIG. 20 indicates the direction of permitted liquid flow in the check valve. Liquid passes through lumen (20-2) of the check valve, pushing the ball of the valve (20-3) down by compressing the valve spring (20-4). Once fluid no longer flows, spring (20-4) pushes ball (20-3) back to seal the valve. The check valve of FIG. 20 is inserted into the check valve adapter component of FIG. 19; FIG. 20A depicts the check valve adapter (19-0) with the check valve (20-1) installed. The check valve adapter containing the check valve is inserted into a container to form a variable loading volume container as in FIG. 21. The container (21-1) is made of a flexible material which can withstand pressurization up to about 5 kbar, preferably up to about 10 kbar and optionally is substantially impermeable to oxygen. The container (21-1) of the variable loading volume device can be injection molded in a single cavity mold (such as those available from PTG Global Inc., Orange County, California) using materials such as Styrolux® 684D. The material of construction is not limited to Styrolux®, and could be further adjusted to modulate oxygen permeability. The container contains liquid sample (21-2). In one embodiment, the variable loading volume device can hold a liquid volume of up to 1.2 mls, and can be used in the volume range of 150-1200 uL. Adapter (21-3) with check valve (21-4) is inserted into the top of the container (21-1); the check valve is oriented so that air and fluid in the container can flow up and out of the container, but fluid is blocked from flowing down and into the container. As the adapter is pushed down into the container, air is forced out of the check valve; the concave bottom of the adapter ensures that as much air as possible is forced out before liquid sample begins to be forced out of the container. The variable loading volume device as depicted can then be subjected to high pressure.

Device Embodiments. Solution Exchange (Solution Mixing) Devices

In another embodiment, the high-pressure device comprises a plurality of compartments, where the contents of the compartments can be kept separate or can be mixed together. Such devices are designated as solution exchange or solution mixing devices. When treating a liquid sample at high pressure, the contents of the containers can be mixed to alter the chemical solution conditions of the liquid sample. The chemical solution conditions which can be changed include, but are not limited to, any one or more of pH, salt concentration, reducing agent concentration, oxidizing agent concentration, chaotrope concentration, concentration of arginine, concentration of surfactant, preferentially excluding compound concentration, ligand concentration, the concentration of any compounds originally present in the liquid sample, or addition of an additional reactant or reagent to add to the solution. In another embodiment, the chemical solution conditions are changed by adding an additional reagent or reactant to the liquid sample. Such a reagent or reactant may comprise an enzyme inhibitor, a drug, a small organic molecule (of molecular weight below about 1000 Daltons), or a protein derivatization reagent.

Container(s)-in-container embodiment: In one such embodiment comprising a plurality of compartments where the contents of the compartments can be kept separate or can be mixed together, the high-pressure device comprises a primary compartment enclosing one or more secondary compartments, where the one or more secondary compartments can be opened without opening the primary compartment, whereby the contents of the one or more secondary compartments are released into contact with the contents of the primary compartment. An example of this embodiment is shown in FIG. 8A and FIG. 8B. A variable loading volume container, such as the variable loading volume container of FIG. 6 or FIG. 21, is used as the primary compartment, while one or more secondary containers (8-1) are placed within the interior (6-5) of the variable loading volume container. (It should be noted that the variable loading volume container is used simply as an example; any of the other devices of the invention, such as the constant loading volume container, can be used as the primary compartment.) One end of the secondary container(s) is sealed. A magnetic disk (8-3) is placed on the other end of the secondary container(s), which will have an axle built which passes through one wall or side of the secondary container(s), through the center of the disk, and into the facing wall or side of the secondary container(s). The disk should be designed with a tolerance so as to fit as precisely as possible inside the secondary container. The disk is designed to freely rotate on the axle, effectively opening and closing the secondary container(s) in a manner analogous to a conventional butterfly valve. A chamfer or beveled edge is used to enable free rotation of the magnetic disk with as tight a tolerance as possible on the rear of the disk. This design enables the magnetic disk to freely rotate, while providing an effective seal when the disk is in the closed position. When this design is used, it is preferable to maintain the primary container in a position such that the secondary container or containers are in a vertical position, in order for gravity to assist in maintaining the magnetic disk in its closed position. The switch is actuated by electric coils placed in a vertical and horizontal fashion around the exterior of the high pressure vessel in which the primary compartment (which contains the secondary compartment(s)) is placed. The horizontal coils are essentially parallel to the magnetic disks within the pressure vessel, in order to generate a magnetic field which maintains the magnetic disk in the closed or sealed position. This is depicted in FIG. 8A. As pressure vessels are commonly made of stainless steel, the coils are designed with the appropriate number of loops, gauge thickness, and current to enable the generation of a magnetic field strong enough penetrate into the interior of the pressure vessel. Alternatively, the pressure vessel could be made out of a material that does not attenuate the magnetic field as much as steel or other such ferromagnetic materials. Another arrangement of electric coils for control of the magnetic disks involves placing coils around the sample rack in a horizontal and vertical manner. In this design, the magnetic field would not have to penetrate the steel walls of the pressure vessel; however, the wires carrying the current would have to run into the interior of the pressure vessel. This can be accomplished by fabricating the base of a sealing plug of a conventional pressure vessel out of an insulating ceramic, rather than steel.

When the magnetic disks are to be kept in the closed position, the horizontal field is turned on and the horizontal field is turned off, maintaining the magnetic disk in the horizontal position and sealing the solution contents of the secondary container from those of the primary container. To enable solution exchange, current in the horizontal and vertical coils is manipulated in the appropriate manner (e.g., turning off the current in the horizontal coils and turning on the current in the vertical coils) to open the disk as depicted in FIG. 8B, allowing the contents of the secondary container(s) (contained in interior space (8-2) of the secondary container) to contact the contents of the primary container (contained in interior space (6-5) of the primary container). The disk can be employed to generate mixing action; current in the horizontal and vertical coils is alternated, with alternating current, to generate a rotating electromagnetic field and flip the magnetic disk. This opens the contents of the secondary container to the primary container, while the motion of the disk enables convection and solution exchange. In a variation, the cap(s) on the secondary container(s) can be controlled by drive shafts which enter the high pressure chamber through appropriately sealed openings into the high pressure chamber, and which also pass through the primary container through appropriate seals.

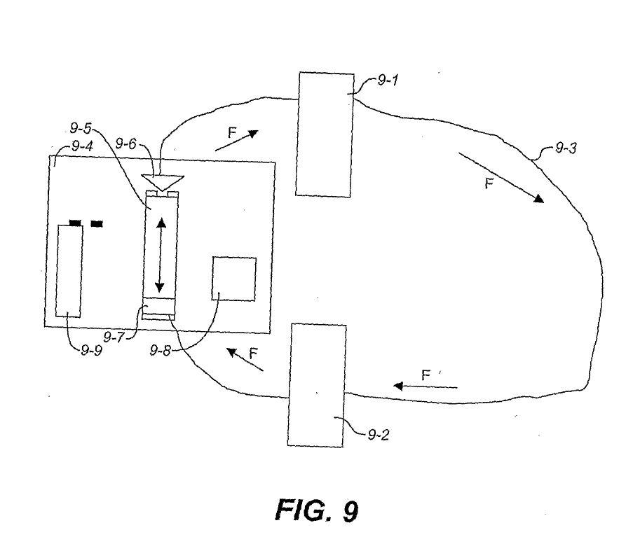

Flow-loop embodiment: In another such embodiment comprising a plurality of compartments where the contents of the compartments can be kept separate or can be mixed together, the high-pressure device comprises at least two compartments connected by flow paths, where the compartments and the flow paths form a closed circular loop with at least one pump. An example of this embodiment is shown in FIG. 9. A liquid sample is placed in the “dissociation” chamber (9-1), while a second solution is placed in the “refolding” chamber (9-2). Additional dissociation chambers, refolding chambers, and flow paths can be added as desired. The device is then placed in the pressure chamber (not shown) and pressurized. When mixing of the liquid sample with the second solution is desired, a piston pump (9-5) is turned on, circulating the liquids through the closed circular loop (9-3). The piston (9-7) can be made of a magnetized material, enabling control of the pump rate by a magnetic field. Microprocessor-controlled battery-powered coils can be placed inside the pressure chamber, along with the chambers and flow loop, in order to control the piston pump. (The microprocessor (9-8) and battery (9-9) are preferably embedded in an epoxy block (9-4) to reduce pressure transfer to the microprocessor itself.) Alternatively, the arrangement of metal coils for control of the secondary compartment metal disk in the primary container/secondary container device can be used to control the piston. In yet another variation, the device can be controlled by drive shafts which enter the high pressure chamber through appropriately sealed openings into the high pressure chamber. One or more check valves (9-6) ensure unidirectional flow. While the containers are labeled “dissociation chamber” and “refolding chamber” for ease of understanding of the figure, it will be appreciated that other chemical and biochemical processes can take place in either or both chambers.

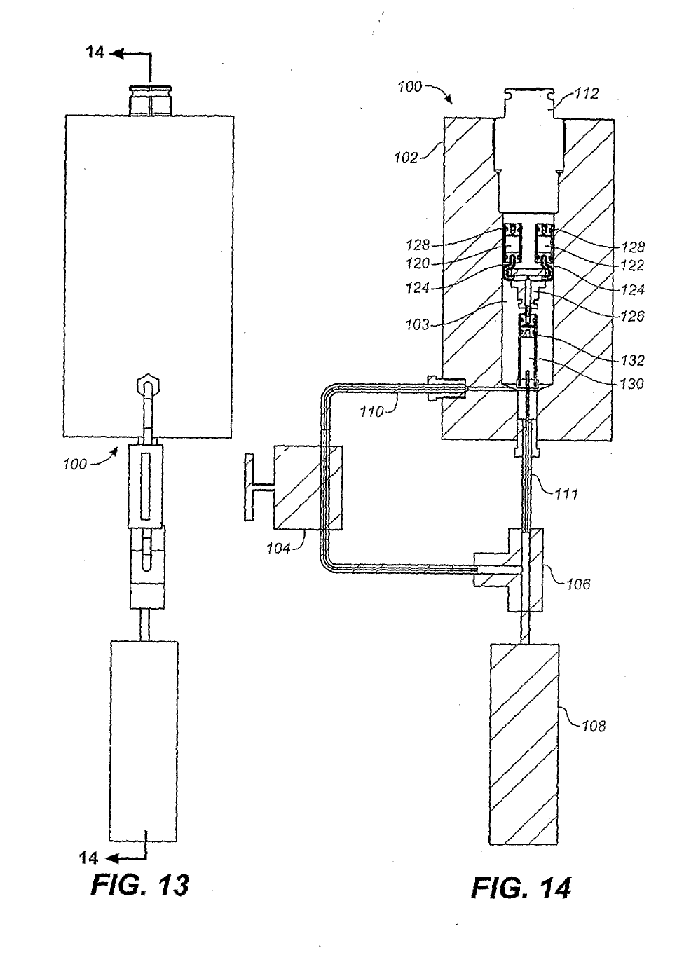

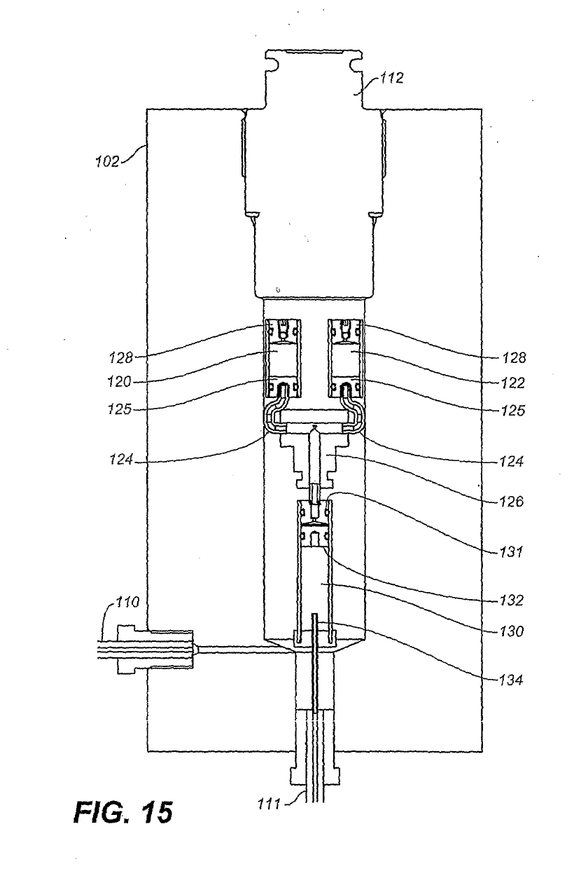

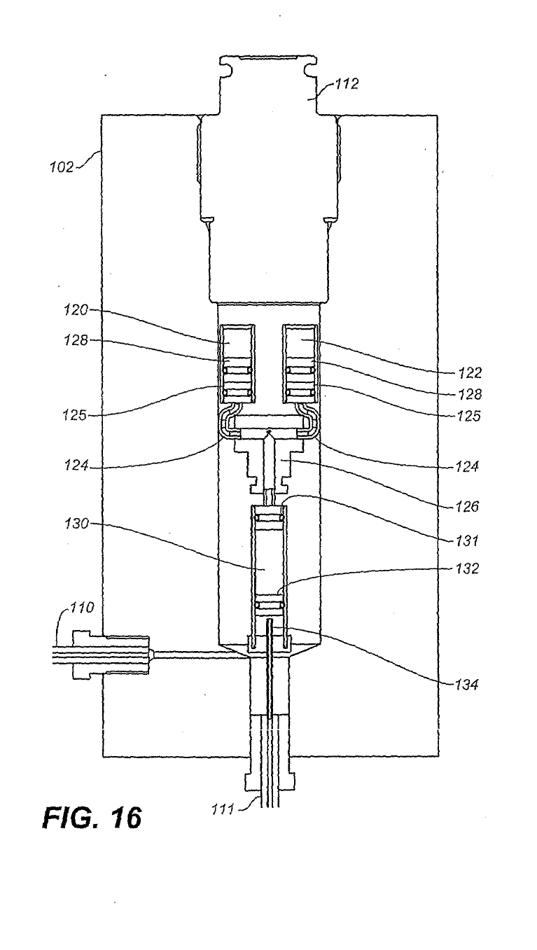

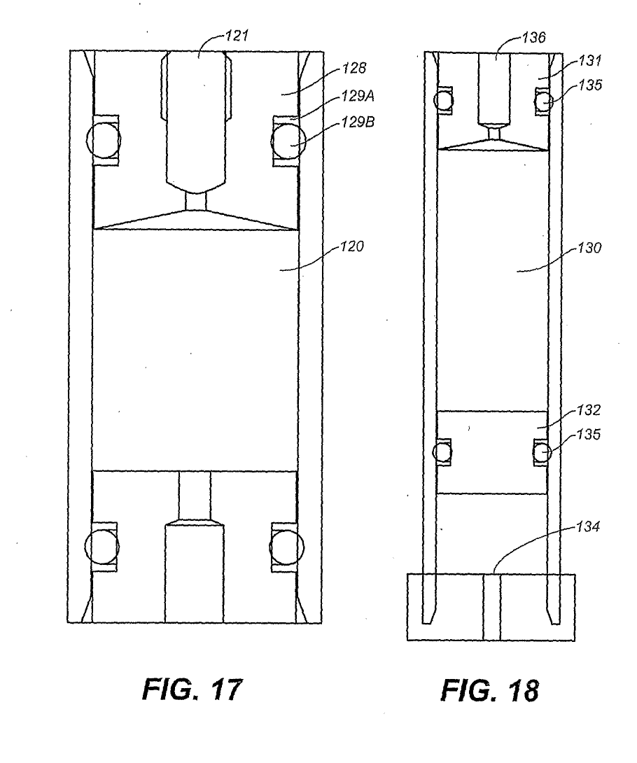

Pre-mix container(s)/receiving (post-mix) container embodiment: In another such embodiment comprising a plurality of compartments where the contents of the compartments can be kept separate or can be mixed together, the high-pressure device comprises a system comprising at least two containers holding liquid samples designated pre-mix containers. The liquid samples usually differ in one or more conditions or compositions, such as salt concentration, pH, etc. (The liquid samples can be the same if desired.) The system also comprises at least one additional container designated the receiving container or post-mix container, where the receiving (post-mix) container can be empty prior to transfer or can contain a liquid or solid composition prior to transfer. Such a system (100) is depicted in FIG. 13, and in detailed cross-section in FIG. 14. In FIG. 14, a pressure chamber (102) sealed by plug (112) supports two pre-mix containers, (120) and (122), which contain separate liquid samples. The pre-mix containers are depicted as roughly equal in size in FIG. 14; however, the size of the containers can be varied relative to each other, so that, for example, one pre-mix container could have twice the volume as the other pre-mix container. Also, for simplicity, only two pre-mix containers are depicted, but more pre-mix containers can be used if desired. The pre-mix containers have mobile pistons (128); liquid conduits (124) lead to a mixer (126). The mixer leads to receiving (post-mix) container (130) containing piston (132), which is depicted as flush against the top of the receiving container in FIG. 14. Valve (104), pressure generator (108), and pressure line (110) communicate with the inside (103) of the pressure chamber (102), and can pressurize the inside of the pressure chamber (102) up to, for example, 2,000-2,500 bar. Valve (106), pressure generator (108), and hydraulic line (111) communicate with liquid disposed beneath the piston (132). FIG. 15 shows the pressure chamber (102) in more detail. Hydraulic outlet (134) removes liquid from receiving container (130), causing piston (132) to be pulled away from seal (131), i.e., piston (132) is drawn away from the inlet from mixer (126). This then draws the liquids in pre-mix containers (120) and (122) through mixer (126), where the liquids mix en route to receiving container (130). Pistons (128) are pulled down as liquid exits containers (120) and (122); when pre-mix containers (120) and (122) are emptied, the pistons (128) rest against seals (125). In another embodiment (not shown), hydraulic pressure can be applied to the external side of pistons (128) to facilitate fluid expulsion from pre-mix containers (120) and (122). FIG. 16 shows the apparatus after the liquid samples in pre-mix containers (120) and (122) has been transferred to receiving container (130). Pistons (128) are flush against seals (125) after fluid expulsion from pre-mix containers (120) and (122). Piston (132) in receiving container (130) has been pushed away from seal (131) to accommodate liquid being transferred to the receiving container. FIG. 17 depicts pre-mix container (120) in more detail. Sample is introduced into the pre-mix container (120) through inlet (121); after introduction of sample, a plug or check valve can then be inserted into inlet (121) to seal the pre-mix container. Piston (128) has an annular indentation (129A) where O-ring (129B) is seated in order to form a seal between the piston and the wall of the container. FIG. 18 depicts the receiving container (130) in more detail. Liquid enters the receiving container through opening (136) in seal (131) (a check valve, not shown can optionally be disposed in opening (136) in order to prevent backflow); an O-ring (135) enhances the seal. Negative hydraulic pressure is applied via opening (134), which pulls piston (132) downwards, which in turn draws the liquid from the pre-mix containers (not shown in FIG. 18) into the receiving container (130). Piston (132) has an O-ring (135) to prevent fluid transfer around the piston.

Pressure chamber (102) can be pressurized so as to generate 2000-2500 bar (higher or lower values, such as 250 bar to 10 kbar, or 1 kbar to 10 kbar, or 1 kbar to 5 kbar, can also be employed) on the liquid samples. Thus the pre-mix containers, the receiving container, and consequently the liquid samples themselves can be maintained at high pressure before, during, and after mixing. The device thus allows for two or more solutions to be treated or incubated separately at high pressure for a first period of time (for example, from about 1 minute to about 1 week, or about 10 minutes to about 48 hours, or about 1 hour to about 48 hours, or about 10 minutes to about 24 hours, or about 1 hours to about 24 hours, or about 10 minutes to about 12 hours, or about 1 hour to about 12 hours, or about 1 hour to about 6 hours). The solutions can then be mixed together; the mixed solutions can be incubated for a second period of time (for example, from about 1 minute to about 1 week, or about 10 minutes to about 48 hours, or about 1 hour to about 48 hours, or about 10 minutes to about 24 hours, or about 1 hours to about 24 hours, or about 10 minutes to about 12 hours, or about 1 hour to about 12 hours, or about 1 hour to about 6 hours). After both incubation periods are complete, the pressure chamber is depressurized, and the solution is removed from the receiving chamber, where it can be analyzed for various properties (such as proper refolding of a protein) and/or used for a desired purpose.

Examples of equipment that can be used include: high pressure generator, PN# 37-5.75-60, High Pressure Equipment Co., Erie, Pa. (in the form of a syringe pump, rated to 60,000 psi); high pressure tubing (PN# 60-9H4-304, High Pressure Equipment Co.); high pressure valves (PN# 60-11HF4, High Pressure Equipment Co.); high pressure glands (PN# 60-2HM4) and collars (PN# 60-2H4 from High Pressure Equipment Co.). The pre-mix containers can be manufactured from quartz Suprasil cylinders (Wilmad Glass, Buena, N.J.); the quartz cylinders can be capped with manufactured stainless steel pistons (High Precision Devices, Boulder, Colo.) which are equipped with O-rings (McMaster-Carr, Aurora, Ohio, PN 9396K16, 2-011, made from silicon rubber) The outlet of the primary chambers is connected to the static mixer through the use of standard HPLC chromatography fittings (PN# F-300-01, F-113, F-126x, 1576, Upchurch, Oak Harbor, Wash.). The mixing device as depicted in the Figures is optional; when a mixing rate higher than simple diffusion is desired, or when thorough mixing is desired, such a mixer can be employed. Static mixers, such as those used in HPLC applications, can be used; these can be obtained from numerous suppliers (for example, Analytical Scientific Instruments, El Sobrante, Calif., static mixer PN# 40200000.5). The outlet of static mixer is connected to the secondary refolding chamber through the use of standard HPLC chromatography fittings (e.g., the Upchurch fittings as previously described).

Stepwise adjustment of solution conditions: In the pre-mix container(s)/receiving (post-mix) container embodiment, it should be noted that the solutions need not be mixed in their entireties in one step; that is, a portion of the solutions in the pre-mix containers can be drawn into the receiving container, followed by continued incubation under pressure of the remaining solutions in the pre-mix containers as well as in the receiving container. In this manner, stepwise adjustment of solution conditions can be implemented. In additional embodiments, the pre-mix containers can have separately actuated valves for addition of different pre-mix solutions at different points in time. Thus, for example, for pre-mix containers designated A, B, C, and D, a liquid sample, such as a protein solution, in pre-mix container A can be incubated for a period of time, then (with valves to A and B open, but valves to C and D closed) the contents of pre-mix containers A and B can be drawn into the receiving container, to alter the original solution conditions of the liquid sample from container A. After a further period of incubation, the valve to pre-mix container C can be opened, and the contents of container C drawn into the receiving container. After yet another period of incubation, the valve to pre-mix container D can be opened, and the contents of container D drawn into the receiving container, followed by still another period of incubation, if desired. This can be implemented with as many pre-mix containers as desired in order to adjust the solution conditions of the liquid sample in a stepwise fashion.

In the flow-loop embodiment, stepwise adjustment of solution conditions can be implemented by having several containers, designated, for example, containers A, B, C, and D. The solutions can be incubated under high pressure for a period of time. Then valves to containers A and B can be opened, allowing flow between those containers (and alteration of the solution conditions of container A as its contents mix with the contents of container B), while valves to containers C and D can be kept in a position where flow by-passes containers C and D during an incubation period. The valves can then be set to allow the contents of container C to be placed into the flow loop (e.g., by shutting off the by-pass shunt around container C, and opening the valves to place container C in the flow loop), where the contents of container C are now mixed with the contents of containers A and B in the flow loop (and alteration of the solution conditions of the solution in the flow loop as its contents mix with the contents of container C), while flow continues to by-pass container D, for another incubation period. Finally, valves can be opened to place container D in the flow loop, while shutting off the by-pass shunt around container D, for yet another adjustment of the solution conditions of the solution in the flow loop as its contents mix with the contents of container D, and yet another incubation period. This can be implemented with as many containers in the flow loop as desired, with appropriate valves and by-pass shunts, in order to adjust the solution conditions of the liquid sample in a stepwise fashion.

These embodiments can be used for refolding of proteins under various conditions. Lin, U.S. Pat. No. 6,583,268, and Li, M. and Z. Su (2002), Chromatographia 56(1-2): 33-38, have suggested refolding proteins at high pH with chaotropes, followed by step-wise reduction of pH, dilution of the protein solution, and ultrafiltration and gel chromatography. Using the high-pressure devices as described above, pressure-modulated refolding (pressures of 250-5000 bar) can be conducted in non-denaturing chaotrope solutions at alkaline pH (near 10.0) and then the pH of the solution can be gradually decreased in step-wise fashion until a value of pH 8.0 is obtained. A rate of 0.2 units per 24 hours, which would be a period of 10 days to lower the pH from 10 to 8, is suggested in U.S. Pat. No. 6,583,268; this rate can be adopted as a general condition, or optimal conditions can be determined on a protein-by-protein basis. The use of high hydrostatic pressure can reduce or remove the need to use high concentrations of chaotropes to promote aggregate dissociation. By combining pressure and chaotrope/pH modulated refolding methods, higher refolding yields are expected to be achieved.

In one embodiment, the invention embraces methods of altering solution conditions under high pressure, comprising the steps of: providing at least one composition in a solution in at least one first container; providing at least one agent for changing solution conditions in at least one additional container, where the contents of the at least one additional container are not in contact with the contents of the at least one first container; placing the containers under high pressure; and causing the contents of the at least one additional container to contact the contents of the at least one first container, wherein the contents of the at least one additional container are caused to contact the contents of the at least one first container over time. In one embodiment, the contents of the at least one additional container are caused to contact the contents of the at least one first container in a continuous manner, whereby the solution conditions of the contents of the first container are changed continuously over a period of time. In another embodiment, the contents of the at least one additional container are caused to contact the contents of the at least one first container in a continuous manner, whereby the solution conditions of the contents of the at least one first container are changed step-wise over a period of time. In one embodiment of this step-wise change in solution conditions, the pH is changed, and the pH of the contents of the first container is at about 9 to about 11, or at about 9.5 to about 10.5, or at about 10. In another embodiment of this step-wise change in solution conditions, the pH of the contents of the first container is at about 9 to about 11, or at about 9.5 to about 10.5, or at about 10, and is lowered to a pH of about 7 to about 8.9, or about 7.5 to about 8.5, or about 8. In another embodiment of the stepwise method, the pH is lowered by about 0.01 to about 2 pH units every approximately 24 hours, or by about 0.1 to about 1 pH unit every approximately 24 hours, or by about 0.1 to about 0.5 pH units every approximately 24 hours, or by about 0.1 to about 0.4 pH units every approximately 24 hours, or by about 0.1 to about 0.3 pH units every approximately 24 hours, or by about 0.2 pH units every approximately 24 hours. Incubation periods before, during, and after the solution condition adjustments can be varied as desired for optimal refolding yields; for example, incubation under high pressure can be carried out for a period of any time from about 1 minute to about 1 week, or about 10 minutes to about 48 hours, or about 1 hour to about 48 hours, or about 10 minutes to about 24 hours, or about 1 hours to about 24 hours, or about 10 minutes to about 12 hours, or about 1 hour to about 12 hours, or about 1 hour to about 6 hours prior to adjustment of solution conditions. For gradual continuous change of solution conditions, the adjustment can be carried out for a period of any time from about 1 minute to about 1 week, or about 10 minutes to about 48 hours, or about 1 hour to about 48 hours, or about 10 minutes to about 24 hours, or about 1 hours to about 24 hours, or about 10 minutes to about 12 hours, or about 1 hour to about 12 hours, or about 1 hour to about 6 hours. For step-wise adjustments of solution conditions, the interval between adjustments can be for a period of any time from about 1 minute to about 1 week, or about 10 minutes to about 48 hours, or about 1 hour to about 48 hours, or about 10 minutes to about 24 hours, or about 1 hours to about 24 hours, or about 10 minutes to about 12 hours, or about 1 hour to about 12 hours, or about 1 hour to about 6 hours. Finally, incubation under high pressure after solution conditions have been adjusted to the desired end conditions can be carried out for a period of any time from about 1 minute to about 1 week, or about 10 minutes to about 48 hours, or about 1 hour to about 48 hours, or about 10 minutes to about 24 hours, or about 1 hours to about 24 hours, or about 10 minutes to about 12 hours, or about 1 hour to about 12 hours, or about 1 hour to about 6 hours.

In the method as described above, the contents of the at least one first container may remain in the first container as the solution conditions are changed, as would be the case with the container-in-container embodiment for solution exchange. Alternatively, all or part of the contents of the at least one first container may no longer be in the first container as the solution conditions are changed, as would be the case with the flow-loop or pre-mix container(s)/receiving (post-mix) container embodiments, in which case the alteration of the contents of the first container is occurring in a location partly or entirely apart from the first container. In such a case, it is understood that reference to changing the solution conditions of the contents of the at least one first container refers to changing the solution conditions of the contents that were originally in the at least one first container (i.e., “the contents of the at least one first container” is understood to read as “the original contents of the at least one first container prior to solution exchange”).

Introduction of Samples into the Sample Compartments

Once a suitable material has been chosen for the body of the device, the samples must be introduced into the sample compartments. The device is adapted to receive liquid samples, and thus a variety of standard methods for liquid transfer can be employed. Hand-held or robotic pipettes, syringes, pumps, and other liquid transfer instruments well-known in the art can be employed. Care should be taken to exclude as much residual air as possible from any of the devices prior to pressurization, which helps prevents material failure and prevents the oxygen contained in the air from being dissolved in the system. The devices can be filled in an inert atmosphere, such as nitrogen or argon, in order to prevent residual air that cannot be excluded from altering the oxygen content of the liquid when pressure is applied.

In certain additional embodiments, prior to loading a liquid sample into the compartment, one or more gases will be sparged through the sample. Such gases include, but are not limited to, relatively unreactive gases such as helium, nitrogen, neon, argon, or krypton, where it is desirable to displace as much dissolved oxygen as possible. Usually rigorous exclusion of oxygen is desired, but in certain circumstances where a higher-than-normal oxygen content is desired in the solution, air or oxygen itself can be sparged through the sample. In yet additional embodiments, vacuum may be applied to the sample in order to de-gas the sample. In yet additional embodiments, sparging with unreactive gas can be followed by vacuum treatment in order to remove as much dissolved oxygen as possible; the sparge-pump cycle can be repeated as necessary.

### Sealing of the Sample Compartments and Sample Introduction

Several of the devices of the invention provide their own seal, e.g., the variable loading volume device, which uses a one-way valve plug for sealing purposes. For devices which do not have their own seal, such as the 96-well plate, sample compartments can be sealed with seals fabricated from silicone, rubber or other material. In one embodiment, the seal material is inert to the contents of the sample well, since the liquid sample may come into contact with the seal during the experiment. When a seal such as rubber is used which is not substantially impermeable to oxygen at high pressure, a second seal which is substantially oxygen-impermeable at high pressure can applied over the first seal to reduce or prevent oxygen mass transfer. The one-way valve plug can be used in a variety of other devices in addition to the variable loading volume device, such as the 96-well plate (where up to 96 one-way valve plugs would be used to seal the compartments).

The sample compartments can be sealed before or after introduction of the liquid sample. If the sample compartment is sealed after the introduction of the liquid sample, then the necessity of penetrating the seal is avoided. However, if the sample compartment is sealed before introduction of the liquid sample, the seal must allow introduction of the sample. A seal made of materials such as rubber or silicone can be pierced with a needle in order to introduce liquid sample; a second needle can be used to vent air from the compartment. The second, venting needle is inserted only to the extent needed to penetrate the seal and minimally extend into the chamber, in order to withdraw as much air as possible. Filling of the chamber is complete once air is completely expelled and liquid begins to be expelled from the chamber.

Since rubber and certain silicones are relatively permeable to oxygen at high pressure, a second sealing layer can be applied in order to prevent mass transfer of oxygen at high pressure. A layer of Mylar® or other suitable material which is substantially oxygen-impermeable at high pressure can be laid down over the first seals.

### Other Applications

It should be noted that, while the high-pressure devices have been discussed above in the context of pharmaceuticals, and in particular for the refolding of proteins, the application of these devices is not limited to the pharmaceuticals or protein refolding. The devices can be used in any applications requiring pressure treatment of samples, particularly liquid samples. For example, Kunugi et al., Langmuir, 15:4056 (1999) studied temperature and pressure responsive behavior of thermoresponsive polymers in aqueous solutions at various pressures. Pressure is well-known to affect chemical reactions; pressure can affect both reaction kinetics (reactions with negative activation volumes are accelerated by higher pressure; see Vaneldik et al., Chemical Reviews 89:549 (1989) and Drljaca et al., Chemical Reviews 98:2167 (1998)) and reaction thermodynamics (transitions which lower system volume are favored by higher pressure; see J. M. Smith et al., Introduction to Chemical Engineering Thermodynamics, New York: McGraw-Hill, 2001).

The invention will be further understood by the following illustrative examples, which are not intended to limit the invention.

### EXAMPLES

### Example 1

### Model Solution Exchange (Solution Mixing) Experiment Using Coomassie Blue Dye

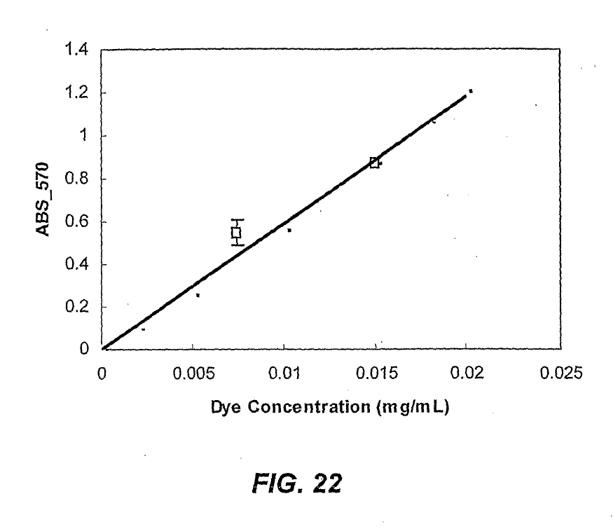

Solution exchange was studied during pressure treatment with the solution mixing device described in FIGS. 13-18. A dilution of a known concentration of Coomassie Blue dye was placed in one pre-mix container (1.0 ml of 0.015 mg/ml dye). In the other pre-mix sample container, 1 ml of pure water was placed. Pressure was slowly increased to 2000 bar. After 10 minutes at this pressure, the high pressure valve connecting to the side inlet of the chamber is closed and the high pressure syringe is withdrawn to modulate the piston flow (a calibration was previously conducted to equate the piston location of the syringe pump relative to piston location of the pre-mix and receiving solution containers). The sample was collected and UV/VIS absorbance measured at 570 nm to determine the final concentration of dye after exchange (FIG. 22). This data was compared to a standard of Coomassie Blue. Three sequential experiments were conducted to determine the extent of mixing that occurred after operating the solution exchange device described in FIGS. 13-18. An absorbance value of 0.55+/−0.5 was measured, corresponding to a dye concentration of 0.0092 mg/ml dye. A 1:1 dilution of the dye solution with the pure water, post-mixing, should result in a dye concentration of 0.0075 mg/ml, with an absorbance of 0.43 at 570 nm (FIG. 22). The study demonstrates that mixing occurred after operating the device three times, with 1.24 volumes of the solution containing Coommassie Blue dye mixing with 0.75 volumes of deionized water. This data demonstrates that solution exchange occurred during pressure treatment.

### Example 2

### Pressure Refolding of Hen Egg White Lysozyme Coupled with Solution Exchange During Pressure Treatment

This example demonstrates that solution exchange during pressure treatment alters the refolding and recovery of native protein from protein aggregates. In previous work, St. John et al. demonstrated that pressure-induced refolding of protein aggregates can be optimized when non-denaturing levels of GdnHCl are present during pressure treatment. St. John et al. showed that lysozyme refolding recoveries increased linearly from ca. 35% at 0.2M GdnHCl to ca. 80% at 2M GdnHCl after incubation at 2000 bar for five days (St John, R. J., J. F. Carpenter, et al. (2002), Biotechnology Progress 18(3): 565-571).

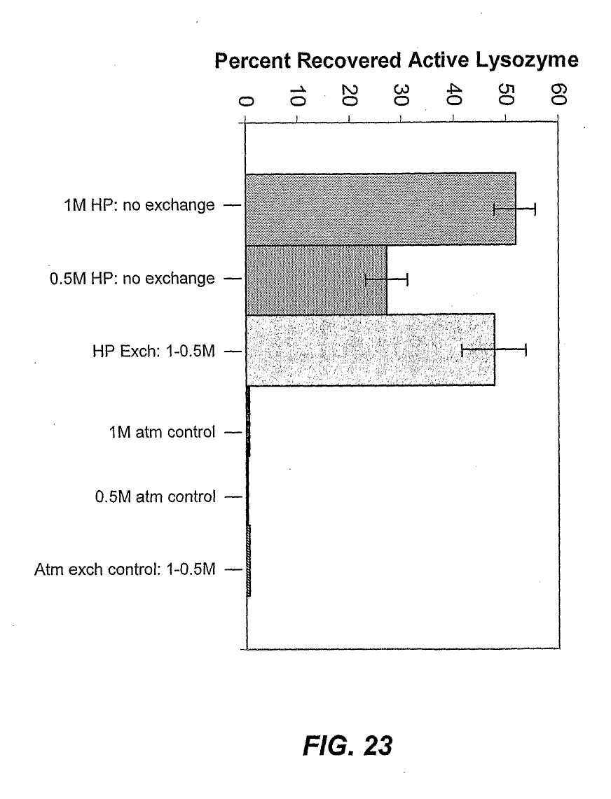

FIG. 23 shows the results from the current lysozyme refolding studies where the GdnHCl concentrations were manipulated both before pressurization to 2000 bar (‘no exchange’ samples) and during pressurization (‘HP-Exch’). (Atmospheric controls were also run, and demonstrated that pressure treatment was needed to refold the lysozyme aggregates.) Lysozyme was refolded with 1M GdnHCl at high pressure (no solution exchange), resulting in a refolding yield of ca. 53%. Lysozyme was also refolded at 0.5M GdnHCl at high pressure, without solution exchange, resulting in a refolding yield of ca. 27%. When lysozyme was refolded at an initial 1M GdnHCl concentration, followed by solution exchange and reduction to a 0.5 M GdnHCl concentration during pressure treatment, a refolding yield of ca. 47% resulted. Thus, while the latter two experiments both had a final concentration of 0.5M GdnHCl, the non-exchanged solution had a much lower refolding yield that the exchanged solution. The non-exchanged lysozyme solution refolded at 1.0M GdnHCl had a higher refolding yield than either of the solutions ending at 0.5M GdnHCl.

High pressure destabilizes hydrophobic and electrostatic contacts but has very little effect on hydrogen bonding. GdnHCl, on the other hand, destabilizes hydrogen bonding. Therefore, the addition of non-denaturing levels of GdnHCl helps facilitate refolding of lysozyme. During the high pressure solution exchange, the initial higher GdnHCl concentration (1M) introduces the lysozyme aggregate to a more favorable environment for aggregate dissociation. Solution exchange under pressure was then completed to bring the final GdnHCl concentration to 0.5M. As previously stated, it can be seen that even though the final solution conditions of both the 0.5 M GdnHCl ‘no exchange’ sample and the solution exchanged sample are the same, refolding was facilitated in the solution exchanged sample by the ability to initially start at the higher 1M chaotrope concentration. The 1M GdnHCl “no exchange” refolding yield emphasizes that lysozyme remains in the native conformation in the presence of 1M guanidine, 2000 bar (Randolph, T. W., M. Seefeldt, et al. (2002), Biochimica Et Biophysica Acta-Protein Structure and Molecular Enzymology 1595(1-2): 224-234). Consequently, refolding yields of lysozyme are not decreased by the presence of the high concentration of chaotrope. Solution exchange during pressure treatment to lower chaotrope-concentrations can be more beneficial towards increasing yields for proteins that are more sensitive to the presence of guanidine HCl. These results show the ability to successfully increase the refolding yield of a protein aggregate using the technique of solution exchange during high pressure treatment.

The experimental conditions used were as follows: An aqueous suspension of aggregated hen egg white lysozyme was placed in one pre-mix container with 50 mM Tris-HCl, 1M GdnHCl, 5 mM GSSG, 2 mM DTT at pH 8.0. A second pre-mix container was filled with 50 mM tris-HCl, 0M GdnHCl, 5 mM GSSG, 2 mM DTT at pH 8.0 containing no protein. The samples were pressurized over a period of 10 minutes to a final pressure of 2000 bar. The protein was kept in the dissolution enhancing buffer for 6 hours, at which point solution exchange was initiated, using the solution exchange device depicted in FIG. 14. The final combined solution (now in the receiving container) remained at 2000 bar for another 6 hours before depressurization. Controls were tested which refolded identical lysozyme aggregates in solutions containing 50 mM Tris-HCl, 0.5 or 1M GdnHCl, 5 mM GSSG, 2 mM DTT at pH 8.0, at pressures of 2000 and 1 bar. The sample was collected from the receiving container and lysozyme catalytic activity was measured by a method similar to the one described by Jolles (Jolles, P. (1962). “Lysozymes from Rabbit Spleen and Dog Spleen.” Methods of Enzymology 5: 137).

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A container for pressure treatment of a liquid sample, comprising at least one compartment for holding the liquid sample, wherein said container is fabricated from a flexible material, where the material can withstand up to 10 kbar of multi-dimensional pressure without breakage or rupture and is optionally substantially impermeable to oxygen at high pressure.

2. The container of claim 1, wherein the container has a variable loading volume at standard pressure.

3. The container of claim 2, wherein the container comprises a cylinder, said cylinder having a first end and a second end; a moveable plug inserted into the first end of the cylinder; and a removable portion affixed to the second end of the cylinder.

4. The container of claim 3, wherein the removable portion is a threaded screw-cap.

5. The container of claim 3, wherein the removable portion is a tip that can be cut off of the container or broken off of the container.

6. The container of claim 2, wherein the moveable plug comprises a check valve.

7. A method of subjecting a sample to high pressure, comprising introducing a sample into the container of claim 1, subjecting the container to high pressure, and reducing the pressure to atmospheric pressure.

8. The method of claim 7, wherein the sample is a solution of an aggregated protein and/or a denatured protein.

9. The container of claim 1, wherein the container is formed from a material selected from the group consisting of polyethyleneterephthalate, high-density polyethylene, polystyrene, and polystyrene-butadiene block copolymers.

10. The container of claim 1, wherein the container has a constant loading volume at standard pressure.

11. A device for solution exchange at high pressure, comprising:
at least one first container holding a first liquid sample;
one or more additional containers holding an additional liquid sample or samples, where the first liquid sample and additional liquid sample or samples can be the same or different,
where the containers are fabricated from materials that can withstand up to 5 kbar of pressure without breakage or rupture and optionally are substantially impermeable to oxygen at high pressure,
and where the liquid sample of the one or more additional containers can be mixed or contacted with the liquid sample of the first container, while both first and additional containers and their respective liquid samples can be maintained at high pressure before, during, and after mixing or contacting.

12. The device of claim 11, wherein when the one or more additional containers comprise two or more additional containers, the contents of the two or more additional containers can be mixed with the contents of the first container either independently of the other two or more additional containers, or in conjunction with the other two or more additional containers.

13. The device of claim 11, wherein:
the at least one first container is a pre-mix container holding a liquid sample (where the liquid samples can be the same or different);
the one or more additional containers holding an additional liquid sample or samples is/are another pre-mix container, where the first liquid sample and additional liquid sample or samples can be the same or different,
and the device further comprises at least one additional receiving container, where the receiving container can be empty prior to transfer or can contain a liquid or solid composition prior to transfer;
where all the containers are fabricated from materials that can withstand up to 5 kbar of pressure without breakage or rupture and optionally are substantially impermeable to oxygen at high pressure;
where the liquid samples in the pre-mix containers holding liquid samples can be transferred into the at least one receiving container whereby the liquid samples can contact and/or mix with each other;
and where the pre-mix containers holding liquid samples, the at least one receiving container, and the liquid samples themselves can be maintained at high pressure before, during, and after contacting and/or mixing.

14. The device of claim 13, further comprising a mixing device interposed in the fluid path between the pre-mix containers holding liquid samples and the at least one receiving container.

15. The device of claim 14, wherein the mixing device is a static mixer.

16. The device of claim 11, where the first container comprises a compartment for holding a first liquid sample, the first container is fabricated from a flexible material that can withstand up to 5 kbar of pressure without breakage or rupture and optionally is substantially impermeable to oxygen at high pressure;
and one or more additional containers, where the one or more additional containers are fabricated from a flexible material that can withstand up to 5 kbar of pressure without breakage or rupture and optionally is substantially impermeable to oxygen at high pressure;
where the one or more additional containers are completely enclosed by the first container, and where the one or more additional containers contains additional liquid samples, which can be the same or different from each other and from the first liquid sample;
where the one or more additional containers can be opened while within the first container, whereby the first liquid sample and additional liquid sample(s) can contact and/or mix.

17. The device of claim 16, wherein:
the one or more additional containers comprise a cap(s) which can be maintained in a closed position, where the cap can be opened without opening the first container;
and while the first container, one or more additional containers, and all liquid samples can be maintained at high pressure before, during, and after opening the cap(s) of the one or more additional containers.

18. The device of claim 17, wherein the cap is also capable of mixing the liquid sample contained in the first container with the liquid samples of the one or more additional containers.

19. The device of claim 17, wherein the cap(s) comprises a magnetized portion, such as a magnetic disk.

20. The device of claim 11, wherein the at least one first container and the one or more additional containers are connected in a flow loop.

21. The device of claim 20, further comprising a check valve whereby fluid can only flow in one direction in the loop.

22. The device of claim 11, wherein one or more solution conditions of the at least one first liquid sample in the at least one first container are changed when the liquid of the at least one first container is mixed and/or contacted with the liquid in the at least one additional container.

23. The device of claim 22, wherein the one or more solution conditions are selected from: pH, salt concentration, reducing agent concentration, oxidizing agent concentration, both reducing agent concentration and oxidizing agent concentration, chaotrope concentration, arginine concentration, surfactant concentration, preferentially excluding compound concentration, ligand concentration, concentration of any compounds originally present in the solution, or addition of another reactant or reagent.

24. A method of altering solution conditions while under high pressure, comprising:
providing at least one first container holding a first liquid sample;
providing one or more additional containers holding an additional liquid sample or samples, where at least one of the at least one first liquid sample and additional liquid sample or samples is different from the remaining samples;
where the containers are fabricated from materials that can withstand up to 5 kbar of pressure without breakage or rupture and optionally are substantially impermeable to oxygen at high pressure,
and mixing or contacting the liquid sample of the one or more additional containers with the liquid sample of the first container, thereby altering the solution conditions of the at least one first liquid sample;
while maintaining high pressure before, during, and after mixing or contacting.

25. The method of claim 24, wherein the one or more solution conditions of the at least one first liquid sample are selected from: pH, salt concentration, reducing agent concentration, oxidizing agent concentration, both reducing agent concentration and oxidizing agent concentration, chaotrope concentration, arginine concentration, surfactant concentration, preferentially excluding compound concentration, ligand concentration, concentration of any compounds originally present in the solution, or addition of another reactant or reagent.

26. The method of claim 25, wherein the one or more solution conditions of the at least one first liquid sample is pH.

27. The method of claim 26, wherein the pH of the at least one first liquid sample is about pH 9 to about pH 11 before solution exchange, and the pH of the at least one first liquid sample is about pH 7 to about pH 8.9 after solution exchange is complete.

28. The method of claim 27, wherein the pH of the at least one first liquid sample is changed in a step-wise fashion.

29. A multi-sample holding device comprising at least two compartments for receiving liquid samples, wherein said device maintains the compartments as substantially closed systems when subjected to high pressure.

30. A multi-sample holding device comprising:
a) a body made from a material that maintains integrity under high pressure; and
b) a plurality of sample compartments in the body, adapted for receiving liquid samples;
wherein the device does not permit significant transfer of liquid sample either between the plurality of sample compartments or between any sample compartment and the surroundings.

31. The device of claim 30, wherein the plurality of sample compartments comprises at least 96 sample compartments.

32. The device of claim 30, wherein the body is formed from a material selected from the group consisting of polyethyleneterephthalate, high-density polyethylene, polystyrene, and polystyrene-butadiene block copolymers.
